(19) United States
(12) Patent Application Publication
(10) Pub. No.: US 2004/0235732 A1
(43) Pub. Date: Nov. 25, 2004

(54) Method for modulating angiogenesis using prokineticin receptor antagonists

(76) Inventors: Qun-Yong Zhou, Irvine, CA (US); Frederick J. Ehlert, Irvine, CA (US)

Correspondence Address: Cathryn Campbell, McDERMOTT, WILL & EMERY, Suite 700, 4370 La Jolla Village Drive, San Diego, CA, 92122, US

(21) Appl. No.: 10/713,567

(22) Filed: Nov. 13, 2003

Related U.S. Application Data
(63) Continuation-in-part of application No. 10/016,481, filed on Nov. 1, 2001.
(60) Provisional application No. 60/245,882, filed on Nov. 3, 2000.

(51) Int. Cl. A61K 38/17; A61K 38/08

(52) U.S. Cl. 514/12; 514/16; 514/17

(57) ABSTRACT

The invention provides methods of modulating angiogenesis by administering an amount of a prokineticin receptor antagonist effective to alter one or more indicia of angiogenesis, wherein the antagonist contains an amino acid sequence at least 80% identical to amino acids to 7 to 77 of SEQ ID NO:3, which includes (a) the 10 conserved cysteine residues of SEQ ID NO:3, and (b) from 0 to 9 of amino acids 78 to 86 of SEQ ID NO:3, wherein amino acids 1 to 6 of the antagonist do not consist of amino acids AVITGA (SEQ ID NO:21). In another embodiment, the antagonist contains an amino acid sequence at least 80% identical to amino acids to 7 to 77 of SEQ ID NO:6, which includes (a) the 10 conserved cysteine residues of SEQ ID NO:6, and (b) from 0 to 4 of amino acids 78 to 81 of SEQ ID NO:6, wherein amino acids 1 to 6 of the antagonist do not consist of amino acids AVITGA (SEQ ID NO:21).

This application is a continuation-in-part of U.S. application Ser. No. 10/016,481, filed Nov. 1, 2001, which claims the benefit of U.S. Provisional Application No. 60/245,882, filed Nov. 3, 2000; and claims the benefit of U.S. Provisional Application No. 60/426,203, filed Nov. 13, 2002, each of which are incorporated herein by reference.

### BACKGROUND OF THE INVENTION

This invention relates generally to angiogenesis-dependent diseases and, more specifically, to modulating angiogenesis to reduce or treat such diseases.

Angiogenesis, the process of new blood vessel development and formation, is a critical component of the body's normal physiology. The process involves migration of vascular endothelial cells into a tissue, followed by condensation of the endothelial cells into vessels. Angiogenesis is essential to a variety of normal body activities, such as reproduction, development and wound repair. Angiogeneis is regulated by a tightly controlled system that includes both angiogenic stimulators and inhibitors. Loss of control of angiogenesis can lead to abnormal formation of blood vessels (neovascularization), which can either cause or contribute to a particular disease or exacerbate an existing pathological condition.

One disease in which abnormal neovascularization has been implicated is cancer. Solid tumor growth and tumor metastasis are both dependent on angiogenesis. It has been shown, for example, that tumors that enlarge to greater than 2 millimeters in diameter must obtain their own blood supply, and do so by inducing growth of new capillary blood vessels. After these new blood vessels become embedded in the tumor, they provide nutrients and growth factors essential for tumor growth as well as a means for tumor cells to enter the circulation and metastasize to distant sites, such as liver, lung and bone.

In addition, aberrant ocular neovascularization has been implicated as the most common cause of blindness and underlies the pathology of approximately 20 eye diseases. Further, in certain previously existing conditions, such as arthritis, newly formed capillary blood vessels invade joints and destroy cartilage. As another example, in diabetes, new capillaries are formed in the retina, invading the vitreous humor and leading to bleeding, which results in blindness. In addition, angiogenic changes have been implicated in ovarian disorders, such as polycystic ovary syndrome. Unfortunately, medical science has not yet provided safe, effective methods for halting angiogenesis that are useful for treating angiogenesis-dependent diseases and disorders in humans.

Thus, there exists a need for methods for reducing angiogenesis. The present invention satisfies this need and provides related advantages as well.

### SUMMARY OF THE INVENTION

The invention provides methods of modulating angiogenesis by administering an amount of a prokineticin receptor antagonist effective to-alter one or more indicia of angiogenesis, wherein the antagonist contains an amino acid sequence at least 80% identical to amino acids to 7 to 77 of SEQ ID NO:3, which includes (a) the 10 conserved cysteine residues of SEQ ID NO:3, and (b) from 0 to 9 of amino acids 78 to 86 of SEQ ID NO:3, wherein amino acids 1 to 6 of the antagonist do not consist of amino acids AVITGA (SEQ ID NO:21).

In another embodiment, the method involves administering an amount of a prokineticin receptor antagonist effective to alter one or more indicia of angiogenesis, wherein the antagonist contains an amino acid sequence at least 80% identical to amino acids to 7 to 77 of SEQ ID NO:6, which includes (a) the 10 conserved cysteine residues of SEQ ID NO:6, and (b) from 0 to 4 of amino acids 78 to 81 of SEQ ID NO:6, wherein amino acids 1 to 6 of the antagonist do not consist of amino acids AVITGA (SEQ ID NO:21).

The PK receptor antagonist used in a method of the invention can contain a substitution, deletion or addition with respect to wild-type amino acids 1 to 6 of prokineticins, such as those referenced as SEQ ID NOS:3 and 6. A PK receptor antagonist can contain, for example 6 or more amino acids N-terminal to the conserved cysteine residue, which can be, for example, MAVITGA (SEQ ID NO:23). A PK receptor antagonist also can contain 5 or fewer amino acids N-terminal to the first conserved cysteine residue, which can be, for example, VITGA (SEQ ID NO:22).

In a PK receptor antagonist used in a method of the invention, the amino acid residues that differ from residues 7 to 77 of SEQ ID NO:3 or SEQ ID NO:6 can be conservative substitutions thereof. In addition, amino acid residues that differ from residues 7 to 77 of SEQ ID NO:3 can be the corresponding residues from SEQ ID NO:6. Likewise, amino acid residues that differ from residues 7 to 77 of SEQ ID NO:6 can be the corresponding residues from SEQ ID NO:3.

A method of the invention for modulating angiogenesis can involve administering a PK receptor antagonist to an endothelial cell, tissue or animal, and can be used to beneficially treat an angiogenesis-dependent disease.

### BRIEF DESCRIPTION OF THE DRAWINGS

FIG. 1A shows a dose-response curve of several prokineticins and prokineticin receptor (PKR) antagonists assayed for their ability to modulate prokineticin receptor 1 (PKR1)-mediated calcium mobilization. FIG. 1B shows a dose-response curve of various prokineticins (PKs) and prokineticin receptor antagonists assayed for their ability to modulate prokineticin receptor 2 (PKR2)-mediated calcium mobilization.

FIG. 2A shows a dose-response curve of PK receptor antagonist MV PK1 (SEQ ID NO:20) assayed for its ability to inhibit PKR1- and PKR2-mediated calcium mobilization in response to either PK1 or PK2. FIG. 2B shows a dose-response curve of PK receptor antagonist Met PK1 (SEQ ID NO:18) assayed for its ability to inhibit PKR1- and PKR2-mediated calcium mobilization in response to either PK1 or PK2. FIG. 2C shows a dose-response curve of PK receptor antagonist MV PK1 (SEQ ID NO:20) assayed for its ability to inhibit PKR1- and PKR2-mediated calcium mobilization in response to either PK1 or PK2.

FIG. 3 shows a dose-response curve of PK receptor antagonist Met PK1 (SEQ ID NO:18) assayed for its ability to inhibit PKR2-mediated calcium mobilization in response to PK1 when the receptor is pretreated with Met PK1.

FIG. 4 shows a dose-response curve of prokineticin receptor antagonist delA-PK1 (SEQ ID NO: 16) assayed for its ability to activate PKR1- and PKR2-mediated calcium mobilization.

FIG. 5A shows a dose-response curve of prokineticin receptor antagonists MetPK1 and MV PK1 assayed for their ability to inhibit PK1-induced cell proliferation. FIG. 5B shows a bar graph indicating that MV PK1 treatment abolishes PK1-induced CHO cell proliferation.

FIG. 6 shows Schild analyses of the antagonistic effects of MV PK1 (A1MPK1) on PKR1 (A) and PKR2 (B) and the antagonistic effects of MetPK1 on PKR1 (C) and PKR2 (D).

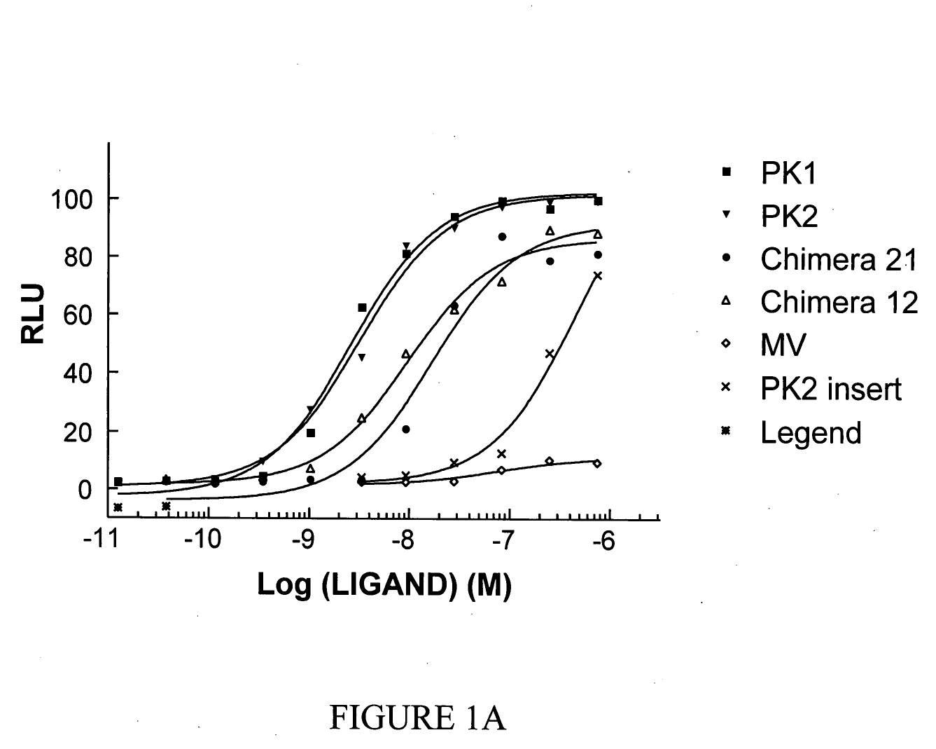

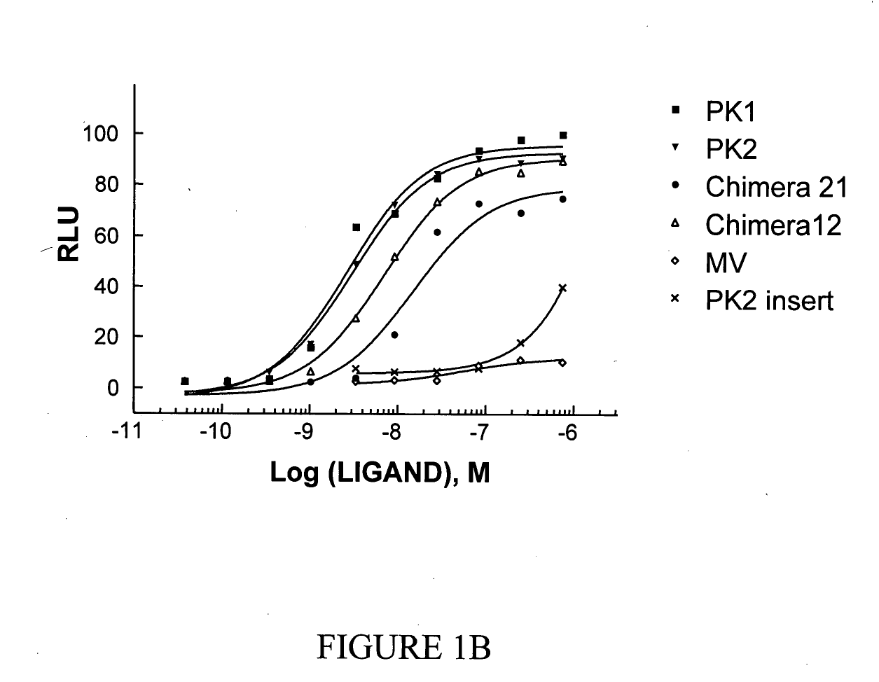

### DETAILED DESCRIPTION OF THE INVENTION

The present invention relates to the determination that prokineticin receptor antagonists can be used to modulate angiogenesis mediated by a prokineticin receptor (PKR). Specifically, PK receptor antagonists that are modified prokineticin polypeptides having structural features described herein have the ability to modulate signaling mediated by prokineticin receptors PKR1 and PKR2. As is described in Example I, calcium mobilization assays were used to show that modified PK polypeptides Met PK1 and MV PK1 inhibit PKR1 and PKR2 activity induced by either PK1 or PK2. These results were corroborated by thymidine incorporation assays using CHO cells expressing PKR1, which showed that modified PK polypeptides MetPK1 and MV PK1 inhibit PKR1-mediated cell growth, as is described in Example III. In addition, as is described in Example IV, Schild analysis was used to show that MetPK1 and MV PK1 function as competitive antagonists of PKR1 and PKR2. In view of the effectiveness of the PK receptor antagonists described herein, and because PK receptors can mediate angiogenesis in a variety of tissues (LeCouter et al., Nature 412:877-884 (2001); Lin et al. J. Biol. Chem. 277:19 (2002)), including endothelium, a PK receptor antagonist having structural features described herein can be used to reduce or inhibit angiogenesis in PK receptor expressing tissues.

A PK receptor antagonist can be used to beneficially modulate angiogenesis in an individual. Methods for modulating angiogenesis have a variety of important applications, including treating individuals having, or who are likely to develop, disorders relating to increased or unwanted angiogenesis, as described in more detail below. Therapeutic methods of modulating angiogenesis involve administering a PK receptor antagonist to an animal, for example to treat an angiogenesis-dependent disease.

Accordingly, the invention provides methods of modulating angiogenesis by administering a PK receptor antagonist, which has a structure described herein below, in an amount effective to alter one or more indicia of angiogenesis.

The methods of the invention involve modulating angiogenesis by administering a PK receptor antagonist described herein below. As used herein, the terms “prokineticin receptor antagonist,” or “PK receptor antagonist,” or “PKR antagonist” refers to a compound that inhibits or decreases normal G-protein coupled signal transduction through a PK receptor. A PK receptor antagonist can act by any antagonistic mechanism, such as by directly binding a PK receptor at the PK binding site, thereby inhibiting binding between the PK receptor and its ligand. A PK receptor antagonist can also act indirectly, for example, by binding a PK. The term “PK receptor antagonist” is also intended to include compounds that act as “inverse agonists,” meaning that they decrease PK receptor signaling from a baseline amount of constitutive signaling activity. A PK receptor antagonist can optionally be selective for PKR1 or PKR2, or alternatively be equally active with respect to both PKR1 and PKR2.

In a method of the invention for modulating angiogenesis, a PK receptor antagonist can be administered to a cell, tissue or animal that expresses a PK receptor. As used herein, the term “prokineticin receptor” or “PKR” refers to a heptahelical membrane-spanning polypeptide that binds to a prokineticin and signals through a G-protein coupled signal transduction pathway in response to prokineticin binding. Prokineticin receptors are believed to couple to the Gα subtype known as Gαq, and thereby mediate intracellular calcium mobilization through a MAPK activation-dependent signaling pathway in response to agonists. A detailed description of prokineticin receptors that can be modulated by a PK receptor antagonist is provided herein below.

A PK receptor antagonist useful in a method of the invention for modulating angiogenesis can be a modified prokineticin (PK). As used herein, the term “prokineticin” or “PK” refers to a peptide that binds to a prokineticin receptor and elicits signaling by the receptor through a G-protein coupled signal transduction pathway.

A PK receptor antagonist can be a modified version of a naturally-occurring amino acid sequence of a PK from any species. For example, a PK receptor antagonist can be a modified mammalian PK, such as a modified human PK1 (SEQ ID NO:3; GenBank Accession No. P58294; also known as endocrine-gland-derived endothelial growth factor or EG-VEGF, TANGO 266, PRO1186 and Zven2; Li et al., supra (2001), LeCouter et al., Nature 412: 877-884 (2001), WO 01/36465, WO 99/63088 and WO 00/52022; a modified human PK2 (GenBank Accession No. Q9HC23; isoform 1, SEQ ID NO:6, Wechselberger et al., FEBS Lett. 462:177-181 (1999) or isoform 2, SEQ ID NO:5; also known as Zven1, Li et al., supra (2001)); a modified mouse PK1 (SEQ ID NO:28; GenBank Accession No. AAM49573); a modified mouse PK2 (SEQ ID NO:29; GenBank Accession No. AAM49572); a modified rat PK1 (SEQ ID NO:30; GenBank Accession No. AAM09104; Masuda et al., supra (2002)); a modified rat PK2 (SEQ ID NO:31; GenBank Accession No. AAM09105; Masuda et al., supra (2002)), a modified rhesus monkey PK2 (SEQ ID NO:34; amino acids 28-108), or a modified PK of another mammalian species, such as other primate, dog, cat, pig, cow, sheep or goat.

A PK receptor antagonist can alternatively be a modified version of a PK of another vertebrate species, such as a snake, frog or toad. For example, the modified PK can be a modified black mamba PK (SEQ ID NO:12; GenBank Accession No. P25687; also known as MIT1; Schweitz et al., FEBS Lett. 461:183-188 (1999)); a modified Bombina variegata frog PK (SEQ ID NO:11; GenBank Accession No. Q9PW66; also known as Bv8; Mollay et al., Eur. J. Pharmacol. 374:189-196 (1999); a modified Bombina maxima toad PK (SEQ ID NO:32; GenBank Accession No. AAN03822), or a modified PK from another vertebrate species, such as an amphibian, reptile, fish or bird.

A PK receptor antagonist also can be a modification of a chimeric PK, such as a modification of a human prokineticin chimera having SEQ ID NO:13 (chimera of PK1 at N-terminus, PK2 at C-terminus) or SEQ ID NO:14 (chimera of PK2 at N-terminus, PK1 at C-terminus).

Exemplary PK receptor antagonists useful in a method of the invention include modified prokineticin polypeptides containing the 10 conserved cysteine residues of wild type prokineticins and the conserved C-terminal residues of wild type prokineticins, but having N-terminal regions different from those of wild-type prokineticins. An N-terminal region of a PK receptor antagonist can include, for example, an addition, deletion or substitution with respect to the six N-terminal amino acids of prokineticins (AVITGA), or an addition or deletion in combination with a substitution, so long as the modified prokineticin exhibits PK receptor antagonistic activity.

A PK receptor antagonist further can be a PK having an N-terminal covalent modification. A number of different reactions can be used to covalently modify a PK, for example, by attaching a moiety to one or more N-terminal amino acid residues. For example, a chemical group on an amino acid, such as an amine group of lysine, a free carboxylic acid group of glutamic or aspartic acid, a sulfhydryl group of cysteine or a moiety of an aromatic amino acids, can be modified using a variety of well known reagents well known to those skilled in the art. One or more selected chemical groups can be modified, for example, by covalent attachment of a moiety. Such moieties include, for example, an organic molecule, such as a dye, or a linker; a detectable moiety, such as a fluorophore or luminescent compound; a macromolecule, such as a polypeptide, nucleic acid, carbohydrate, or lipid, or a modification thereof. Modifications to the N-terminus of a PK amino acid sequence to obtain a PK receptor antagonist include, but are not limited to, the addition of nucleotide or amino acid sequences useful as “tags.” Such tag sequences include, for example, epitope tags, histidine tags, glutathione-S-transferase (GST), and the like, or sorting sequences.

Chemical and enzymatic modifications to a PK to produce a PK receptor antagonist include, but are not limited to the following: replacement of hydrogen by an alkyl, acyl, or amino group; esterification of a carboxyl group with a suitable alkyl or aryl moiety; alkylation of a hydroxyl group to form an ether derivative; phosphorylation or dephosphorylation of a serine, threonine or tyrosine residue; or N- or O-linked glycosylation.

A PK receptor antagonist also can be a non-covalent modification of the N-terminus of a PK. A number of non-covalent interactions can be used to modify a PK. For example, the N-terminus of a PK can be modified by binding to an antibody or other antigen-binding molecule, including a polyclonal and monoclonal antibody, and antigen binding fragments of such antibodies, as well as a single chain antibody, chimeric antibody, bifunctional antibody, CDR-grafted antibody and humanized antibody, and antigen-binding fragments of such antibodies, or any other moiety that can be non-covalently attached to the N-terminus.

A modified prokineticin that is a PK receptor antagonist can be, for example, an N-terminal substitution mutant. Such a mutant can contain any amino acid residues at the six N-terminal amino acids of prokineticins except for AVITGA (SEQ ID NO:21); any amino acid residues at five or fewer amino acids N-terminal to the first conserved cysteine residue; or any amino acid residues at seven or more amino acids N-terminal to the first conserved cysteine residue so long as the mutant has PK receptor antagonistic activity. In one embodiment, a PK receptor antagonist useful in a method of the invention contains the sequence MVITGA (SEQ ID NO:39) N-terminal to the first conserved cysteine residue. In particular, the N-terminal prokineticin mutant designated M VPK1 (SEQ ID NO:20) contains the sequence MVITGA N-terminal to the first conserved cysteine residue, and is an exemplary substitution mutant having antagonistic activity (see Example I).

A PK receptor antagonist of the invention can contain one or more substitutions with respect to a known PK amino acid sequence. Substitutions to PK amino acid sequences, such as SEQ ID NOS:3 or 6, can either be conservative or non-conservative. Conservative amino acid substitutions include, but are not limited to, substitution of an apolar amino acid with another apolar amino acid (such as replacement of leucine with an isoleucine, valine, alanine, proline, tryptophan, phenylalanine or methionine); substitution of a charged amino acid with a similarly charged amino acid (such as replacement of a glutamic acid with an aspartic acid, or replacement of an arginine with a lysine or histidine); substitution of an uncharged polar amino acid with another uncharged polar amino acid (such as replacement of a serine with a glycine, threonine, tyrosine, cysteine, asparagine or glutamine); or substitution of a residue with a different functional group with a residue of similar size and shape (such as replacement of a serine with an alanine; an arginine with a methionine; or a tyrosine with a phenylalanine).

A modified prokineticin that is a PK receptor antagonist can be, for example, an N-terminal addition mutant. Such a mutant can contain 6 or more amino acids N-terminal to the first conserved cysteine residue, such as 7 or more, 8 or more, 9 or more or 10 or more amino acids N-terminal to the first conserved cysteine residue. The 6 or more amino acids N-terminal to the first conserved cysteine can have any amino acid sequence so long as the mutant has PK receptor antagonistic activity. In one embodiment, a PK receptor antagonist useful in a method of the invention contains the sequence MAVITGA (SEQ ID NO:23) N-terminal to the first conserved cysteine residue. In particular, the N-terminal prokineticin mutant designated Met PK1 (SEQ ID NO:18) contains the sequence MAVITGA N-terminal to the first conserved cysteine residue, and is an exemplary addition mutant having antagonistic activity (see Example I).

A modified prokineticin that is a PK receptor antagonist can be, for example, an N-terminal deletion mutant. Such a mutant can contain 5 or fewer amino acids N-terminal to the first conserved cysteine residue, such as 4 or fewer, 3 or fewer or 2 or fewer amino acids N-terminal to the first conserved cysteine residue, including 1 amino acid or no amino acids N-terminal to the first conserved cysteine residue. The 5 or fewer amino acids N-terminal to the first conserved cysteine can have any amino acid sequence so long as the mutant has PK receptor antagonistic activity. In one embodiment, a PK receptor antagonist useful in a method of the invention contains the sequence VITGA (SEQ ID NO:22) N-terminal to the first conserved cysteine residue. The N-terminal prokineticin mutant designated DelA PK1 (SEQ ID NO:16) contains the sequence VITGA N-terminal to the first conserved cysteine and is an exemplary deletion mutant having antagonistic activity.

In one embodiment, a PK receptor antagonist useful in a method of the invention contains an amino acid sequence at least 80% identical to amino acids to 7 to 77 of SEQ ID NO:3, and includes (a) the 10 conserved cysteine residues of SEQ ID NO:3, and (b) from 0 to 9 of amino acids 78 to 86 of SEQ ID NO:3, wherein amino acids 1 to 6 of the antagonist do not consist of amino acids AVITGA (SEQ ID NO:21).

In another embodiment, a PK receptor antagonist useful in a method of the invention contains an amino acid sequence at least 80% identical to amino acids to 7 to 77 of SEQ ID NO:6, and includes (a) the 10 conserved cysteine residues of SEQ ID NO:6, and (b) from 0 to 4 of amino acids 78 to 81 of SEQ ID NO:6, wherein amino acids 1 to 6 of the antagonist do not consist of amino acids AVITGA (SEQ ID NO:21).

The amino acid residues that differ from residues 7 to 77 of SEQ ID NO:3 can be, for example, the corresponding residues from SEQ ID NO:6. Likewise, the amino acid residues that differ from residues 7 to 77 of SEQ ID NO:6 can be, for example, the corresponding residues from SEQ ID NO:3. In an embodiment, a PK receptor antagonist useful in a method of the invention contains amino acids 7 to 77 of SEQ ID NO:3. In another embodiment, a PK receptor antagonist useful in a method of the invention contains amino acids 7 to 77 of SEQ ID NO:6.

A prokineticin receptor antagonist therefore can be an amino acid sequence at least 80% identical to amino acids to 7 to 77 of SEQ ID NO:3 or 6, at least 90% identical to amino acids to 7 to 77 of SEQ ID NO:3, at least 95% identical to amino acids to 7 to 77 of SEQ ID NO:3 or 6, and at least 98% identical to amino acids to 7 to 77 of SEQ ID NO:3 or 6, including an amino acid sequence that is identical to amino acids 7 to 77 of SEQ ID NO:3 or 6.

A PK receptor antagonist useful in a method of the invention will generally have an IC₅₀ that is no more than 2-fold, 5-fold, 10-fold, 50-fold, 100-fold or 1000-fold higher or lower than the EC₅₀ for human PK1 or PK2 in the particular assay. For therapeutic applications described below, a PK receptor antagonist preferably has an IC₅₀, of less than about 10⁻⁷ M, such as less than 10⁻⁸ M, and more preferably less than 10⁻⁹ or 10₋₁₀ M. However, depending on the stability, selectivity and toxicity of the compound, a PK receptor antagonist with a higher IC₅₀, can also be useful therapeutically. As is described in Examples I, III, and IV, and in Table 1, below, PK receptor antagonists Met PK1 and MV PK1 have nanomolar antagonist activity with respect to both PKR1 and PKR2, in the presence of either PK1 or PK2.

TABLE 1
Antagonistic Activity of PK mutants
(Calcium Mobilization Assay)

| Receptor | Ligand | Met PK1 (nM) | MV PK1 (nM) |
|---|---|---|---|
| PKR1 | PK1 | 9 | 6 |
| PKR2 | PK2 | 30 | 29 |
| PKR2 | PK1 | 15 | 16 |
| PKR1 | PK2 | 90 | 110 |

In a method of the invention, a PK receptor modulated by a PK receptor antagonist can be contained within a naturally occurring cell or a cell that expresses recombinant PK receptor. A PK receptor that can be modulated by a PK receptor antagonist described herein above can have the naturally-occurring amino acid sequence of a PK receptor from any species, or can contain minor modifications with respect to the naturally-occurring sequence. For example, such a PK receptor can be a mammalian PK receptor, such as human PKR1 (SEQ ID NO:24; GenBank Accession No. AAM48127; also called GPR73, fb41a, hZAQ, hGPRv21 and EG-VEGF receptor-1; Lin et al., J. Biol. Chem. 277:19276-19280 (2002), Masuda et al., Biochem. Biophys. Res. Commun. 293:396-402 (2002), WO 00/34334, WO 01/48188 and WO 01/16309); human PKR2 (SEQ ID NO:25; GenBank Accession No. AAM48128; also known as 15E, hRUP8 and hZAQ2; Lin et al., supra (2002), Masuda et al., supra (2002), WO 98/46620, WO 01/36471 and WO 02/06483); chimpanzee PKR2 (SEQ ID NO:36); squirrel monkey PKR2 (SEQ ID NO:38); mouse PKR1 (SEQ ID NO:26; GenBank Accession No. AAM49570; Cheng et al., Nature 417:405-410 (2002) and WO 02/06483); mouse PKR2 (SEQ ID NO:27; GenBank Accession No. AAM49571; Cheng et al., supra (2002) and WO 02/06483); rat PKR1 (WO 02/06483); rat PKR2 (WO 02/06483); monkey PKR2 (also known as AXOR8; WO 01/53308); bovine PKR1 (Masuda et al., supra (2002), or a PKR of another mammalian species, such as other primate, dog, cat, pig, sheep or goat; or a PKR of another vertebrate species, such as an amphibian, reptile, fish or bird.

In a method of the invention, a PK receptor modulated by a PK receptor antagonist can contain minor modifications with respect to a naturally-occurring PK receptor can contain one or more additions, deletions, or substitutions of natural or non-natural amino acids relative to the naturally-occurring polypeptide sequence, so long as the receptor retains PK receptor signaling activity in response to PK. Such a modification can be, for example, a conservative change, wherein a substituted amino acid has similar structural or chemical properties, for example, substitution of an apolar amino acid with another apolar amino acid, substitution fo a charged amino acid with another amino acid of similar charge, and the like. Such a modification can also be a non-conservative change, wherein a substituted amino acid has different but sufficiently similar structural or chemical properties so as to not adversely affect the desired biological activity. Further, a minor modification can be the substitution of an L-configuration amino acid with the corresponding D-configuration amino acid with a non-natural amino acid. In addition, a minor modification can be a chemical or enzymatic modification to the polypeptide, such as replacement of hydrogen by an alkyl, acyl, or amino group; esterification of a carboxyl group with a suitable alkyl or aryl moiety; alkylation of a hydroxyl group to form an ether derivative; phosphorylation or dephosphorylation of a serine, threonine or tyrosine residue; or N- or O-linked glycosylation.

To determine or confirm that a PK receptor antagonist has PK receptor antagonistic activity, a variety of well-known assays can be employed. Such assays include both PK receptor signaling assays and ligand binding assays.

Signaling assays to identify or confirm the activity of PK receptor antagonists are known in the art. Because PK receptors are Gαq-coupled receptors, signaling assays typically used with other Gαq-coupled GPCRs can be used to determine PK receptor signaling activity. Gαq-coupled GPCRs, when bound to ligand, activate phospholipase C (PLC), which cleaves the lipid phosphatidylinositol 4,5-bisphosphate (PIP2) to generate the second messengers inositol 1,4,5-trisphosphate (IP3) and diacylglycerol (DAG). These second messengers increase intracellular Ca²⁺ concentration and activate the MAP kinase cascade. The change in activity of PLC, or in abundance of downstream messengers, is a reflection of GPCR activation.

The specificity of Gα subunits for cell-surface receptors is determined by the C-terminal five amino acids of the Ga. Thus, if it is desired to assay a GPCR signaling pathway other than a typical Gαq pathway, a chimeric Gα containing the five C-terminal residues of Gαq and the remainder of the protein corresponding to another Gα can be expressed in a cell such that the PK receptor is coupled to a different signaling pathway (see, for example, Conklin et al., Nature 363:274-276 (1993), and Komatsuzaki et al., FEBS Letters 406:165-170 (1995)). For example, a PK receptor can be coupled to a Gαs or Gαi, and adenylate cyclase activation or inhibition assayed by methods known in the art.

Depending on the Gα and the assay system, GPCR signals that can be determined include, but are not limited to, calcium ion mobilization; increased or decreased production or liberation of arachidonic acid, acetylcholine, diacylglycerol, cGMP, cAMP, inositol phosphate and ions; altered cell membrane potential; GTP hydrolysis; influx or efflux of amino acids; increased or decreased phosphorylation of intracellular proteins; and activation of transcription of an endogenous gene or promoter-reporter construct downstream of any of the above-described second messenger pathways. An exemplary calcium mobilization assay for PK receptor signaling in response to prokineticins is shown in Example I and an exemplary thymidine incorporation assay for PK receptor growth signaling in response to prokineticins is shown in Example III.

A variety of cell-based GPCR signaling assays, including assays performed in bacteria, yeast, baculovirus/insect systems and mammalian cells, are reviewed, for example, in Tate et al., Trends in Biotech. 14:426-430 (1996). More recently developed GPCR signaling assays include, for example, AequoScreen, which is a cellular aequorin-based functional assay that detects calcium mobilization (LePoul et al., J. Biomol. Screen. 7:57-65 (2002)); MAP kinase reporter assays (Rees et al., J. Biomol. Screen. 6:19-27 (2001); and fluorescence resonance energy transfer (FRET) based PLC activation assays (van der Wal, J. Biol. Chem. 276:15337-15344 (2001)). Several examples of PK receptor signaling assays are described in Lin et al., supra (2002) and in Masuda et al., supra (2002).

A PK receptor antagonist can be tested to determine whether it antagonizes PK binding to a PK receptor using a variety of well-known assays. Competitive and non-competitive binding assays for detecting ligand binding to a receptor are described, for example, in Mellentin-Micelotti et al., Anal. Biochem. 272:182-190 (1999); Zuck et al., Proc. Natl. Acad. Sci. USA 96:11122-11127 (1999); and Zhang et al., Anal. Biochem. 268;134-142 (1999). Examples of PK receptor binding assays are described in Lin et al., supra (2002) and in Masuda et al., supra (2002).

Depending on the intended application, the skilled person can determine an appropriate form for the PK receptor, such as in a live animal, a tissue, a tissue extract, a cell, a cell extract, or in substantially purified form. For example, for confirming the antagonistic activity of a PR receptor antagonist in receptor binding or signaling assays, the PK receptor will typically be either endogenously expressed or recombinantly expressed at the surface of a cell.

Cells that endogenously express a PK receptor are well known in the art, and include, for example, M2A7 melanoma cells (available from American Type Culture Collection as ATCC CRL-2500), M2 melanoma cells (Cunningham et al., Science 255;325-327 (1992)) and RC-4B/C pituitary tumor cells (ATCC CRL-1903)(see US 20020115610A1). Other cells that endogenously express a PK receptor include, for example, ileal and other gastrointestinal cells (see U.S. Pat. No. 20020115610A1), endothelial cells such as BACE cells (Masuda et al., supra (2002)) and endothelial cells from adrenal cortex, choroid plexus, aorta, umbilical vein, brain capillary, microvessels of endocrine pancreas and dermal microvasculature; endocrine cells (Lin et al., supra (2002)), neural stem and progenitor cells, including cells in the subventricular zone of the lateral ventricle, the olfactory bulb/olfactory ventricle, the dentate gyrus of the hippocampus, and the inner nuclear layer of the retina.

The methods of the invention involve administering an amount of a PK receptor antagonist effective to modulate one or more indicia of angiogenesis. As used herein, the term “angiogenesis” means the process of formation of blood vessels, including de novo formation of vessels such as that arising from vasculogenesis as well as that arising from branching and sprouting of existing vessels, capillaries and venules. Angiogenesis encompasses the cellular processes of proliferation, migration, differentiation and survival of endothelial cells that occurs during the development of new blood vessels. The term is intended to cover angiogenesis as it occurs during normal development, wound healing, and reproductive functions, as well as angiogenesis that occurs in pathological conditions.

As used herein, the term “effective” when used in reference to an amount of a PK receptor antagonist used to alter one or more indicia of angiogenesis, means an amount of a PK receptor antagonist sufficient to alter a read-out corresponding to a particular index of angiogenesis by at least about 10%, such as at least 25%, 50%, 2-fold, 5-fold, 10-fold, 50-fold, 100-fold or more, in comparison to a control.

As used herein, the term “modulating” means causing an alteration in the amount of angiogenesis compared to a control level of angiogenesis. Such alterations include an increase or decrease in the rate or amount of angiogenesis. The rate of amount of angiogenesis in a tissue can be modulated by promoting or inhibiting cellular processes that contribute to blood vessel formation, such as cell proliferation, differentiation, migration, and survival. A PK receptor antagonist can modulate angiogenesis by reducing or inhibiting signaling of a PK receptor, thereby reducing or inhibiting downstream events resulting from PK receptor signaling, such as cell proliferation, differentiation, migration, and/or survival. Therefore, the ability of a PK receptor antagonist to modulate angiogenesis can be assessed with respect to a cell capable of undergoing proliferation, differentiation, migration or survival in response to PK, a cell undergoing a process of blood vessel formation, in a tissue undergoing or capable of undergoing blood vessel formation, or an animal in which blood vessel formation is occurring or is capable of occurring.

As used herein the term “index” or “indicia” when used in reference to angiogenesis means an observable sign or indication of angiogenesis. An index of angiogenesis can be observed in a cell, tissue or animal because alterations in cellular functions that affect angiogenesis, such as endothelial cell proliferation, migration, differentiation and survival, can be observed in endothelial cells capable of undergoing such alterations, in tissues containing such endothelial cells, as well as in animals containing such tissues. Exemplary indicia of angiogenesis include cellular indicia of angiogenesis, such as cell proliferation, cell migration, cell differentiation, and cell survival; tissue indicia of angiogenesis, such as extent of capillary formation, and complexity of capillary formation, and animal indicia of angiogenesis, such as metastasis; secondary tumor formation; tumor growth; and the like. Cellular indicia of angiogenesis also can be observed in a tissue or animal; likewise, tissue indicia of angiogenesis can be observed in an animal.

Modulation of angiogenesis can be evidenced following administering a PK receptor antagonist to a cell, tissue or animal. Considerable insight in the molecular and cellular biology of angiogenesis has been obtained by in vitro studies using endothelial cells, isolated from either capillaries or large vessels (see, for example, Cockerill, et al., Int. Rev. Cytol., 159:113-160 (1995); Fan, et al., “In Vivo Models of Angiogenesis” In Tumor Angiogenesis, ed. Bicknell, R. et al. Oxford University press, 5-18, (1997). Most steps in the angiogenic cascade can be analyzed in vitro, including endothelial cell proliferation, migration and differentiation (see, for example, Montesano, R. et al. EXS, 61:129-136, (1992)).

Endothelial cells proliferate in response to an angiogenic stimulus during neovascularization. Therefore, proliferation of endothelial cells in response to an angiogenic stimulus, such as a PK, is a useful index of angiogenesis. As a read-out for angiogenesis, proliferation studies can be based, for example, on cell counting; thymidine incorporation; or immunohistochemical staining for cell proliferation, such as by measurement of PCNA; and on determining activity of a signaling molecule having an activity that correlates with proliferation, such as MAP kinase activity. Methods for determining MAP kinase activity are well known to those skilled in the art and kits for determining MAP kinase activity are commercially available, for example, from Upstate Cell Signaling Solutions; Waltham, Mass., and New England Biolabs; Beverly, Mass. Proliferation studies also can be based on determining a reduction in cell death, such as by terminal deoxynucleotidyl transferase-mediated dUTP nick end labeling or Tunel assay. An exemplary proliferation assay is a bovine capillary endothelial cell proliferation assay. Briefly, bovine capillary endothelial cells stimulated with bFGF can be used to determine the efficacy of a PK receptor antagonist in reducing angiogenesis. The cells are cultured in the presence or absence of an angiogenesis stimulating agent, such as a PK, and in the presence or absence of a PK receptor antagonist. The extent of proliferation is measured following an about 72 hour culture period to determine the effect of the PK receptor antagonist on cell growth and therefore, on angiogenesis. The bovine capillary endothelial cell proliferation assay is well known in the art and is described in, for example, PCT publication WO 97/15666, which is incorporated herein by reference. The extent of endothelial cell proliferation in the presence of the antagonist compared to control treatment in the absence of the antagonist inversely correlates with the activity and/or efficacy of the PK receptor antagonist. Thus, endothelial cell proliferation is an index of angiogenesis that can be negatively altered, or reduced, by administering a PK receptor antagonist. Similarly, endothelial cell migration, differentiation and survival are indices of angiogenesis that can be negatively altered, or reduced, by administering a PK receptor antagonist.

The process of endothelial cell migration through the extracellular matrix towards an angiogenic stimulus is also a critical event required for angiogenesis. Therefore, migration of endothelial cells in response to an angiogenic stimulus is a useful index of angiogenesis. As a read-out for angiogenesis, cell migration can be examined, for example, in a Boyden chamber, which consists of an upper and lower well separated by a membrane filter. In a typical format, a chemotactic solution, such as a solution containing a PK, is placed in a lower well, cells are added to an upper well, and after a period of incubation, cells that have migrated toward the chemotactic stimulus are counted on the lower surface of the membrane. Cell migration can also be studied by making a “wound” in a confluent cell layer and calculating the number of cells that migrate and the distance of migration of the cells from the edge of the wound (Fan, et al., supra (1997)). Using any cell migration assay, the ability of a PK receptor antagonist to alter migration, and thereby alter angiogenesis can be determined. The extent of endothelial cell migration in the presence of the antagonist compared to control treatment in the absence of the antagonist inversely correlates with the activity and/or efficacy of the PK receptor antagonist.

As a read-out for angiogenesis, differentiation can be induced in vitro by culturing endothelial cells in different ECM components, including two- and three-dimensional fibrin clots, collagen gels and matrigel (Benelli, R. et al., Int. J. Biol. Markers, 14:243-246, (1999)). The extent of cell differentiation in the presence of the antagonist compared to control treatment in the absence of the antagonist inversely correlates with the activity and/or efficacy of the PK receptor antagonist.

A commercially available in vitro angiogenesis kit, such as Chemicon's In Vitro Angiogenesis Assay Kit (Chemicon International, Temecula, Calif.), in which endothelial cells in solution are placed on top of a gel, allowing the cells to align and form tube-like structures that can be readily observed under a light microscope, also can be used to determine the ability of a PK receptor antagonist to modulate angiogenesis.

Advantages of the above-described in vitro systems include the possibility to control the different parameters, such as the spatial and temporal concentration of angiogenic mediators, such as PK and PK receptor antagonists, the ability to study individual steps in the angiogenic process, and the lower cost, as compared to in vivo experiments. A PK receptor antagonist that alters an indicia of angiogenesis in vitro, such as cell proliferation, survival, migration, or differentiation, can be tested in an in vivo animal model if desired. A PK receptor antagonist that does not alter an index of angiogenesis can have activity in vivo, and thus can also be tested in an in vivo model if desired. Advantages of the below-described in vivo systems include the ability to observe the effect of a PK receptor antagonist within a more complex system and an enhanced ability to predict the effect of an antagonist in an animal, including a human.

A number of ex vivo and in vivo angiogenesis model bioassays are well known and widely used. A model system that includes endocrine gland endothelium, such as endothelial tissues from steroidogenic glands, can be highly responsive to prokineticin and thus can be useful in a method of the invention. Such model systems include, for example, intra-ovarian, intra-testis, intra-adrenal and intra-placental delivery of a PK receptor antagonist in the presence and/or absence of a PK. For an example of such intra-organ delivery, see LeCouter et al., Nature 412:877-884 (2001). In addition, other model systems that can express prokineticin receptors at levels relatively lower than that observed in steroidogenic glands also can be useful in a method of the invention. Such model systems include, for example, rabbit corneal pocket, chick chorioallantoic membrane (“CAM”), rat dorsal air sac and rabbit air chamber bioassays. For a review, see, Blood et al., Biochem. et Biophys. Acta 1032:89-118 (1990).

In the CAM assay, fertilized chick embryos are cultured in Petri dishes. The assay is typically performed as follows. Briefly, 3 day old chicken embryos with intact yolks are separated from the egg and placed in a petri dish. After 3 days of incubation a methylcellulose disc containing a PK receptor antagonist to be tested is applied to the CAM of individual embryos. After 48 hours of incubation, the embryos and CAMs are observed to determine whether endothelial growth has been inhibited. As with the in vitro assays described above, the extent of endothelial cell growth compared to control treatment inversely correlates with the activity and/or efficacy of the PK receptor antagonist. This method is described, for example, in O'Reilly, et al., Cell 79:315-328 (1994), and in U.S. Pat. No. 5,753,230, both of which are incorporated herein by reference.

In the rabbit corneal pocket assay, polymer pellets of ethylene vinyl acetate copolymer (“EVAC”) are impregnated with test substance and surgically implanted in a pocket in the rabbit cornea approximately 1 mm from the limbus (Langer et al., Science 193:707-72 (1976)). To test the ability of a PK receptor antagonist to modulate angiogenesis, either a piece of carcinoma or some other angiogenic stimulant is implanted distal to the polymer 2 mm from the limbus. In the opposite eye of each rabbit, control polymer pellets that are empty are implanted next to an angiogenic stimulant in the same way. In these control corneas, capillary blood vessels start growing towards the tumor implant in 5-6 days, eventually sweeping over the blank polymer. In test corneas, the directional growth of new capillaries from the limbal blood vessel towards the tumor occurs at a reduced rate and is often inhibited such that an avascular region around the polymer is observed. This assay can be quantitated by measurement of the maximum vessel lengths, for example, with a stereospecific microscope. The extent and complexity of capillary formation compared to control treatment inversely correlates with the activity and/or efficacy of the PK receptor antagonist.

The ability of a PK receptor antagonist to modulate angiogenesis also can be determined in vivo using animal models known in the art. For example, animal models for tumor growth and metastasis are applicable for determining the ability of a PK receptor antagonist to reduce or prevent angiogenesis-dependent disease. Briefly, tumor growth can be induced in an animal model by, for example, injecting metastatic tumors into the animal and determining the extent of lung colonization or secondary tumor formation in the presence or absence of a PK receptor antagonist. The extent of lung colonization or secondary tumor function inversely correlates with the activity and/or efficacy of the a PK receptor antagonist. Similar assays can be employed using solid tumors and measuring the size or growth rate of the tumor as an indicator of a PK receptor antagonist activity and/or efficacy. Exemplary tumors that can be used for determining the ability of a PK receptor antagonist to modulate angiogenesis include standard animal tumor models; tumors of endocrine organs, such as thyroid, adrenal gland, pancreas, ovary, uterus, testis and other steroidogenic organs and tissues; vascularized tumors and tumors of vascular origin, including polyomavirus middle T-transformed or chemically induced hemangiosarcomas, hemangioendotheliomas overexpressing FGF-2 and Kaposi's Sarcoma. For a description of tumor-bearing animal models see, for example, U.S. Pat. No. 5,753,230 and PCT publication WO 97/15666 and U.S. Pat. No. 5,639,725. Other animal models are known to those skilled in the art and can similarly be used to determine the effect of a PK receptor antagonist on reducing the extent of tumor growth or metastasis.

As described above, a PK receptor antagonist can be administered to a tumor bearing animal to determine the ability of the antagonist to modulate angiogenesis, compared to a control. A decrease in the rate or extent of tumor growth, or a disappearance of the tumor correlates with the ability of a PK receptor antagonist to reduce angiogenesis and with efficacy against progression of an angiogenesis-dependent disease.

Subcutaneous implantation of various artificial sponges (for example, polyvinyl alcohol, gelatin) in animals has been used frequently to study angiogenesis in vivo. In this method, compounds to be evaluated are either injected directly into the sponges or incorporated into ELVAX or hydron pellets, which are placed in the center of the sponge. Neovascularization of the sponges can be assessed either histologically, morphometrically (vascular density), biochemically (hemoglobin content) or by measuring the blood flow rate in the vasculature of the sponge using a radioactive tracer. See, for example, McCarty et al. International Journal of Oncology, 21:5-10 (2002).

Other examples of in vivo assays for determining the ability of a PK receptor antagonist to modulate angiogenesis are models of ischemia-associated iris neovascularization, for example in primates, and retinal neovascularization, for example, in mouse.

Any of the above-described in vitro, ex vivo and in vitro assays for determining the ability of a PK receptor antagonist to modulate angiogenesis can involve comparison of a test sample, which can be, for example, a cell, tissue, or animal, to a control. One type of a “control” is a sample that is treated identically to the test sample, except the control is not exposed to the PK receptor antagonist. Another type of “control” is a sample that is similar to the test sample, except that the control sample does not express a PK receptor, or has been modified so as not to respond to a PK.

The methods of the invention can be used in vitro, ex vivo, or in vivo for determination of the ability of a PK receptor antagonist to reduce angiogenesis; for determination of an therapeutically effective dosage; and can be used in vivo for a desired therapeutic effect. For in vitro testing in cells, any endothelial cell expressing a PK receptor and capable of producing an index of angiogenesis can be used. Exemplary endothelial cells include endothelial cells from adrenal cortex, choroid plexus, gastrointestinal tract, aorta, umbilical vein, brain capillary, microvessels of endocrine pancreas and dermal microvasculature and endothelial cells from any fenestrated tissue. For ex vivo and in vivo testing, any cell, tissue or animal model system containing a PK receptor and capable of producing an observable index of angiogenesis in response to PK known in the art can be used. For example, the method can be practiced in a suitable animal model systems prior to testing in humans, including, but not limited to, rats, mice, chicken, cows, monkeys, rabbits, and the like.

The methods of the invention can involve administering a PK receptor antagonist to prevent or treat a variety of angiogenesis-dependent diseases. As used herein, the term “administering” when used in reference to a PK receptor antagonist means providing to or contacting a cell, tissue or animal with the PK receptor antagonist. The term encompasses administering a PK receptor antagonist in vitro or ex vivo, as to a cell or tissue, which can be a cell or tissue removed from an animal or a cell or tissue placed in or adapted to culture; as well as in vivo, as to an animal. Modes of administering a PK receptor antagonist are described in detail herein below. As used herein, the term “angiogenesis-dependent” when used in reference to a disease means a disease in which the process of angiogenesis or vasculogenesis sustains or augments a pathological condition. Angiogenesis is the formation of new blood vessels from pre-existing capillaries or post-capillary venules. Vasculogenesis results from the formation of new blood vessels arising from angioblasts, which are endothelial cell precursors. Both processes result in new blood vessel formation and are included within the meaning of the term angiogenesis-dependent diseases.

In one embodiment, the methods of the invention for modulating angiogenesis are useful for reducing or preventing cancer. Reducing or preventing angiogenesis can slow or prevent tumor development and progression because tumorigenesis depends upon angiogenesis for a supply of blood to provide nutrients to the growing tumor and to remove waste products. Moreover, as is described in Example III, PK receptor antagonists of the invention are effective in reducing cell proliferation as indicated by their ability to inhibit thymidine uptake in mammalian cells expressing PKR1.

Prokineticins induce proliferation, migration and morphological changes in endothelial cell types, such as endothelial cells from aorta, umbilical vein, adrenal cortex and dermal microvasculature (see, for example, LeCouter et al., Nature Medicine 8(9):913-917 (2002)). Therefore, in one embodiment, the methods of the invention for reducing angiogenesis can be used to treat endocrine organ cancers and other proliferative and angiogenic diseases of endocrine organs, as described in more detail below.

The methods of the invention for modulating angiogenesis also can be used to reduce or prevent tumor metastasis. Angiogenesis is involved in metastasis in at least two ways. First, vascularization of a tumor allows tumor cells to enter the blood stream and to circulate throughout the body. Second, once tumor cells have arrived at the metastatic site, angiogenesis is required for growth of the new tumor. Both of these stages of metastasis can be reduced or prevented by modulating angiogenesis using a method of the invention.

In another embodiment, the methods of the invention for modulating angiogenesis are useful for reducing or preventing endocrine disorders characterized by excessive angiogenesis, such as ovarian cyst disorders including polycystic ovary syndrome, and ovarian hyperstimulation syndrome.

The methods of the invention are also useful for reducing angiogenesis in other angiogenesis-dependent diseases. Such diseases include several eye diseases, many of which lead to blindness, in which ocular neovascularization occurs in response to the diseased state. Exemplary ocular disorders include diabetic retinopathy, neovascular glaucoma, ocular tumors, ocular neovascular disease, age-related macular degeneration, corneal graft rejection, neovascular glaucoma, retrolental fibroplasia, uveitis, retinopathy of prematurity, macular degeneration, eye diseases associated with choroidal neovascularization and eye diseases associated with iris neovascularization.

Other angiogenesis-dependent diseases include rheumatoid arthritis; osteoarthritis; psoriasis; myocardial angiogenesis; plaque neovascularization; telangiectasia; hemophiliac joints; angiofibroma; wound granulation; chronic inflammation, including ulcerative colitis, Crohn's disease, and Bartonellosis; atherosclerosis; hemangioma; delayed wound healing; granulations; hypertrophic scars; scleroderma; trachoma, and vascular adhesions. Adverse effects of certain hereditary diseases, including Osler-Weber-Rendu disease, and hereditary hemorrhagic telangiectasia are also caused at least in part by angiogenesis and thus are amenable to treatment using the claimed methods.

The methods of the invention for modulating angiogenesis also can be applied to contraceptive methods. Angiogenesis occurs during ovulation and implantation of a blastula after fertilization. Reducing angiogenesis in the ovary and uterus thus can be used to prevent ovulation and implantation.

Additionally, the methods of the invention for modulating angiogenesis can be applied to reducing the development of fenestrae in endothelial cells. Fenestrae are highly permeable to fluid and small solutes and are thought to facilitate large exchange of materials between interstitial fluid and plasma.

As is described in Example III, PK1 effectively promotes growth of CHO cells that express PKR1, while PK receptor antagonists of the invention inhibit this PK1-induced cell growth. Therefore, a PK receptor antagonist of the invention can be used to reduce or prevent cell growth in the context of cell proliferation disorders in addition to angiogenesis, such as cancer, restenosis, and fibrosis. Cancer refers to a class of diseases characterized by the uncontrolled growth of aberrant cells, including all known cancers, and neoplastic conditions, whether characterized as malignant, benign, soft tissue or solid tumor. Exemplary cancers that can be treated using the claimed methods are malignant solid tumors including, but not limited to, tumors of endocrine organs, such as ovary, testis, adrenal cortex, thyroid gland, pancreas, uterus, placenta and prostate; glioblastoma, melanoma and Kaposi's sarcoma, tumors of lung, mammary, and colon; epidermoid carcinoma, neuroblastoma, retinoblastoma, rhabdomyosarcoma, Ewing sarcoma, and osteosarcoma; as well as non-malignant tumors, including, but not limited to, acoustic neuroma, neurofibroma, trachoma and pyogenic granuloma.

A PK receptor antagonist used in a method of the invention for modulating angiogenesis can be formulated and administered in a manner and in an amount appropriate for the condition to be treated; the weight, gender, age and health of the individual; the biochemical nature, bioactivity, bioavailability and side effects of the particular compound; and in a manner compatible with concurrent treatment regimens. An appropriate amount and formulation for a particular therapeutic application in humans can be extrapolated based on the activity of the compound in the ex vivo and in vivo angiogenesis assays described herein.

The therapeutically effective dosage for reducing or preventing angiogenesis in vivo can be extrapolated from in vitro assays using a PK receptor antagonist, or a combination of a PK receptor antagonist with other angiogenesis inhibiting factors. The effective dosage is also dependent on the method and means of delivery. As a non-limiting example, in some applications, as in the treatment of angiogenesis-dependent diseases of the skin or eyes, such as psoriasis or diabetic retinopathy, a PK receptor antagonist can be delivered in a topical formulation. In other applications, as in the treatment of solid tumors, a PK receptor antagonist can be delivered, for example, by means of an injection and biodegradable, polymeric implant. In further applications, as in a contraceptive method, a PK receptor antagonist can be delivered, for example, orally and by implant. Those skilled in the art will be able to determine an appropriate route of delivery of a PK receptor antagonist to be used in the methods of the invention for modulating angiogenesis.

The total amount of a PK receptor antagonist can be administered as a single dose or by infusion over a relatively short period of time, or can be administered in multiple doses administered over a more prolonged period of time. Additionally, the compound can be administered in a slow-release matrice, which can be implanted for systemic delivery at or near the site of the target tissue. Contemplated matrices useful for controlled release of compounds, including therapeutic compounds, are well known in the art, and include materials such as DepoFoam™, biopolymers, micropumps, and the like.

A PK receptor antagonist can be administered to an animal by a variety of routes known in the art including, for example, intracerebrally, intraspinally, intravenously, intramuscularly, subcutaneously, intraorbitally, intracapsularly, intraperitoneally, intracisternally, intra-articularly, orally, intravaginally, rectally, topically, intranasally, or transdermally.

Generally, a PK receptor antagonist can be administered to an animal as a pharmaceutical composition comprising the compound and a pharmaceutically acceptable carrier. The choice of pharmaceutically acceptable carrier depends on the route of administration of the compound and on its particular physical and chemical characteristics. Pharmaceutically acceptable carriers are well known in the art and include sterile aqueous solvents such as physiologically buffered saline, and other solvents or vehicles such as glycols, glycerol, oils such as olive oil and injectable organic esters. A pharmaceutically acceptable carrier can further contain physiologically acceptable compounds that stabilize the compound, increase its solubility, or increase its absorption. Such physiologically acceptable compounds include carbohydrates such as glucose, sucrose or detrains; antioxidants, such as ascorbic acid or glutathione; chelating agents; and low molecular weight proteins (see for example, “Remington's Pharmaceutical Sciences” 18th ed., Mack Publishing Co. (1990)).

For applications that require the compounds to cross the blood-brain barrier, or to cross cell membranes, formulations that increase the lipophilicity of the compound can be useful. For example, the compounds of the invention can be incorporated into liposomes (Gregoriadis, Liposome Technology, Vols. I to III, 2nd ed. (CRC Press, Boca Raton Fla. (1993)). Liposomes, which can contain phospholipids or other lipids, are generally nontoxic, physiologically acceptable and metabolizable carriers that are relatively simple to make and administer. Other approaches for formulating a compound such that it crosses the blood-brain barrier are known in the art and include the use of nanoparticles, which are solid colloidal particles ranging in size from 1 to 1000 nm (Lockman et al., Drug Dev. Ind. Pharm. 28:1-13 (2002)), and peptides and peptidomimetics that serve as transport vectors (Pardridge, Nat. Rev. Drua Discov. 1:131-139 (2002).

For applications in which is it desirable to administer a PK receptor antagonist locally to the area in need of treatment, a PK receptor antagonist can be provided, for example, by local infusion during surgery; topical application, such as in conjunction with a wound dressing after surgery; by injection; by means of a catheter; by means of a suppository; and by means of an implant, such as a porous, non-porous, or gelatinous material, including membranes, such as sialastic membranes, or fibers. For topical application, a PK receptor antagonist can be combined with a carrier, such as, for example, an ointment, cream, gel, paste, foam, aerosol, suppository, pad or gelled stick. A PK receptor antagonist also can be admixed in a ophthalmologically acceptable excipient such as buffered saline, mineral oil, vegetable oils such as corn or arachis oil, petroleum jelly, Miglyol 182, alcohol solutions, or liposomes or liposome-like products.

For oral administration applications, a PK receptor antagonist can be formulated in tablet or capsule form, which can contain, for example, any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; or a glidant such as colloidal silicon dioxide. When the dosage unit form is a capsule, it can contain, in addition to material of the above type, a liquid carrier such as a fatty oil. In addition, dosage unit forms can contain various other materials which modify the physical form of the dosage unit, for example, coatings of sugar, shellac, or other enteric agents. Suppositories generally contain active ingredient in the range of 0.5% to 10% by weight; oral formulations generally contain 10% to 95% active ingredient.

To enhance the modulation of angiogenesis, more than one therapeutic approach or composition can be provided to an individual. For example, PK receptor antagonist that modulates angiogenesis can be used in conjunction with conventional therapies for the disorder or condition being treated. As a non-limiting example, for treating cancer, a PK receptor antagonist can be administered either alone or in conjunction with another cancer therapy. Exemplary cancer therapies with which PK receptor antagonist administration can be combined include but are not limited to chemotherapy, radiation therapy, and surgical intervention. Such treatments can act in a synergistic manner, with the reduction in tumor mass caused by the conventional therapy increasing the effectiveness of the PK receptor antagonist, and vice versa. Non-limiting examples of anti-cancer drugs that are suitable for co-administration with a PK receptor antagonist are well known to those skilled in the art of cancer therapy and include aminoglutethimide, amsacrine (m-AMSA), azacitidine, asparaginase, bleomycin, busulfan, carboplatin, carmustine (BCNU), chlorambucil, cisplatin (cis-DDP), cyclophosphamide, cytarabine HCl, dacarbazine, dactinomycin, daunorubicin HCl, doxorubicin HCl, erythropoietin, estramustine phosphate sodium, etoposide (V16-213), floxuridine, fluorouracil (5-FU), flutamide, hexamethylmelamine (HMM), hydroxyurea (hydroxycarbamide), ifosfamide, interferon alpha, interleukin 2, leuprolide acetate (LHRH-releasing factor analogue), lomustine (CCNU), mechlorethamine HCl (nitrogen mustard), melphalan, mercaptopurine, mesna, methotrexate (MTX), mitoguazone (methyl-GAG, methyl glyoxal bis-guanylhydrazone, MGBG), mitomycin, mitotane (o. p′-DDD), mitoxantrone HCl, octreotide, pentostatin, plicamycin, procarbazine HCl, semustine (methyl-CCNU), streptozocin, tamoxifen citrate, teniposide (VM-26), thioguanine, thiotepa, vinblastine sulfate, vincristine sulfate, and vindesine sulfate.

As another non-limiting example, a PK receptor antagonist can be administered together with Vascular Endothelial Growth Factor (VEGF) inhibitors and therapies that reduce VEGF receptor activity, including gene therapy, to treat an angiogenesis-dependent disease. Exemplary VEGF inhibitors include, but are not limited to, compounds that block VEGF receptor signaling, such as anti-VEGF receptor antibodies (Genentech; South San Francisco, Calif.); SU5416 and SU6668 (SUGEN; South San Francisco, Calif.), PTK787/ZK 22584 (Novartis; East Hanover, N.J.); compounds that inhibit VEGF production, such as Interferon-alpha; and compounds that inhibit VEGF receptor production, such as antisense molecules (Kamiyama et al., Cancer Gene Therapy 9, 197-201 (2002)).

It is understood that modifications which do not substantially affect the activity of the various embodiments of this invention are also included within the definition of the invention provided herein. Accordingly, the following examples are intended to illustrate but not limit the present invention.

### EXAMPLE I

### Prokineticin Receptor Antagonists Reduce Prokineticin Receptor-Mediated Calcium Mobilization

This example shows the ability of prokineticin receptor antagonists to reduce prokineticin receptor 1 (PKR1)-mediated calcium mobilization and prokineticin receptor 2 (PKR2)-mediated calcium mobilization.

To determine whether various modified prokineticins (PKs) have the ability to modulate PK receptor function, the modified prokineticins were tested for their ability to function as agonists or antagonists in PK receptor-mediated calcium mobilization assays. Shown in Table 2 below are the structures of several of the modified prokineticins tested.

An aequorin-based luminescent assay for measuring mobilization of intracellular Ca²⁺ was performed essentially as described in Liu et al., supra, (2002). Chinese hamster ovary (CHO) cells stably expressing photoprotein aequorin and hPKR1 or hPKR2 were used for this assay. Briefly, the cells was charged in Opti MEM containing 30 μM reduced glutathione and 8 μM of coelenterazine cp at 37° C. for 2 hours. The cells were then detached by typsinization, spun down, rinsed once with PBS, recentrifuged, resuspended and maintained in Hank's Balanced Salt Solution(HBSS) plus 10 mM HEPES (pH7.5) and 0.1% BSA at about 5×10⁵ cells/ml. Measurements were recorded using a Monolight 2010 luminometer (Analytical Luminescence Laboratory).

For agonist assays, 100 ul of cells were injected into 20 ul of ligand, and luminescence was recorded for 15 seconds. For antagonist assays, 100 ul of cells were injected into a mixture of 20 ul antagonist and 100 ul PK1 or PK2 (10 nM), and luminescence was recorded for 15 seconds. For antagonist assays with preincubation, 100 ul of PK1 or PK2 (10 nM) was injected into a mixture of 20 ul antagonist and 100 ul cells, which were incubated at RT for 1 hour.

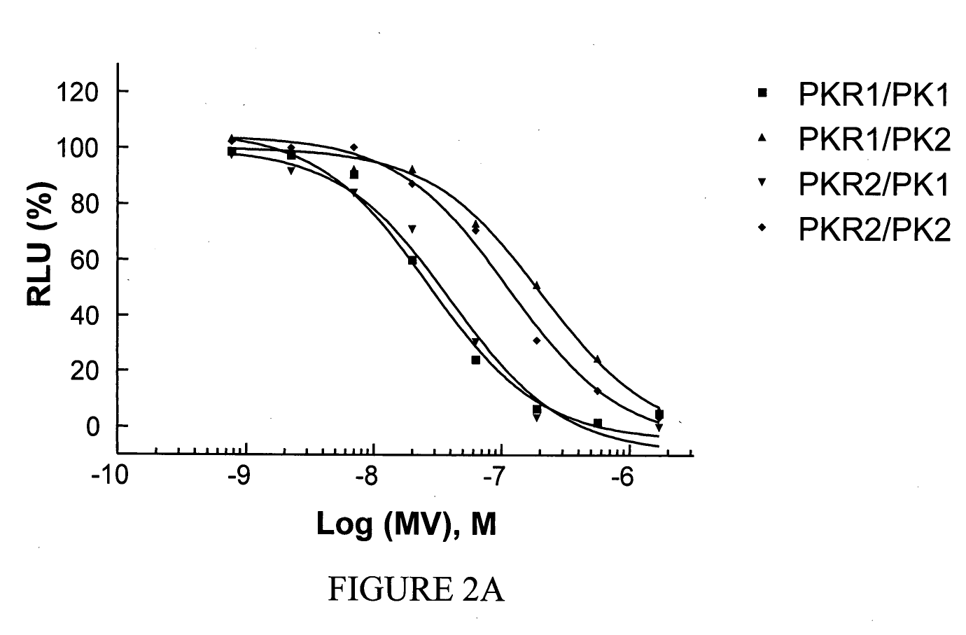

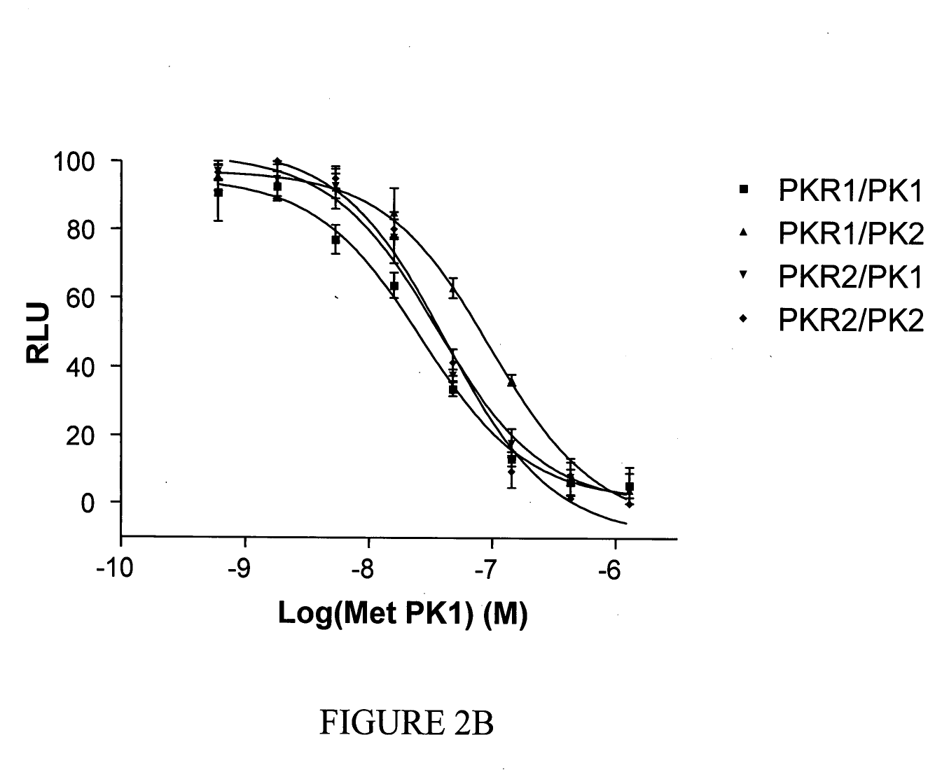

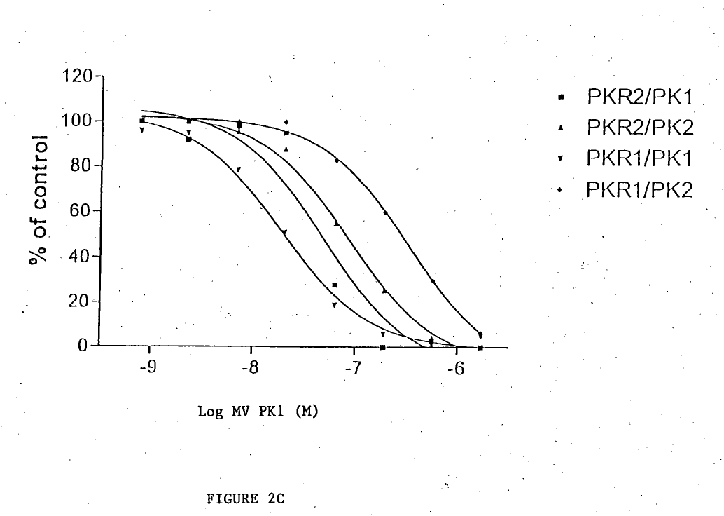

FIG. 2A shows a dose-response curve of PK receptor antagonist MV PK1 (SEQ ID NO:20) assayed for its ability to inhibit PKR1- and PKR2-mediated calcium mobilization in response to either PK1 or PK2. FIG. 2B shows a dose-response curve of PK receptor antagonist Met PK1 (SEQ ID NO:18) assayed for its ability to inhibit PKR1- and PKR2-mediated calcium mobilization in response to either PK1 or PK2. FIG. 2C shows a dose-response curve of PK receptor antagonist MV PK1 (SEQ ID NO:20) assayed for its ability to inhibit PKR1- and PKR2-mediated calcium mobilization in response to either PK1 or PK2.

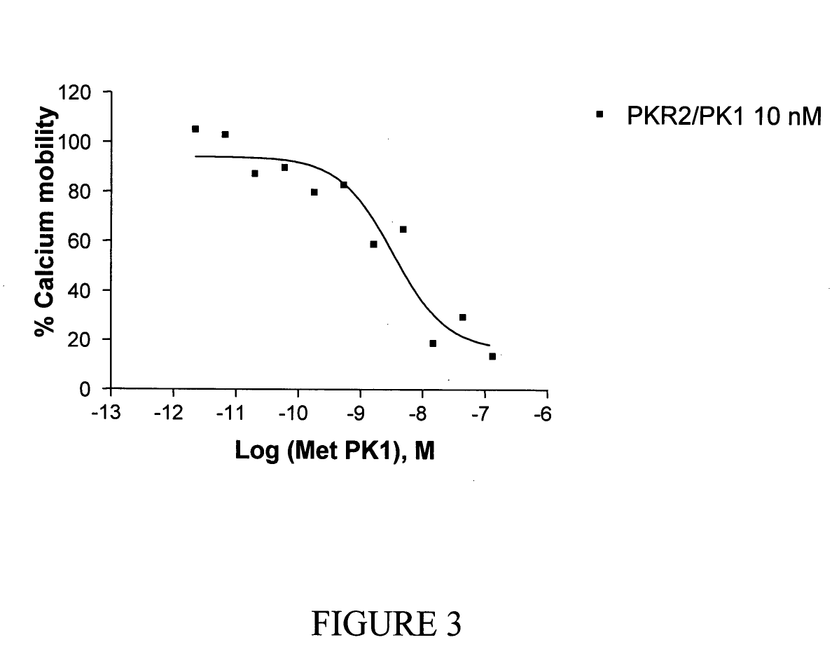

To determine whether pretreatment of a PK receptor with a modified prokineticin alters the ability of the modified prokineticin to modulate PK receptor function, Met PK1 was preincubated with receptor for 1 hour prior to stimulation of the receptor with ligand (PK1, 10 nM). FIG. 3 shows a dose-response curve of PK receptor antagonist Met PK1 (SEQ ID NO:18), which indicates that Met PK1 is more potent in antagonizing PK1 effect in a pretreatment regimen. The IC₅₀ for Met PK1 with pretreatment is 3.3 nM, whereas the IC₅₀ for Met PK1 in the absence of pretreatment is 36 nM.

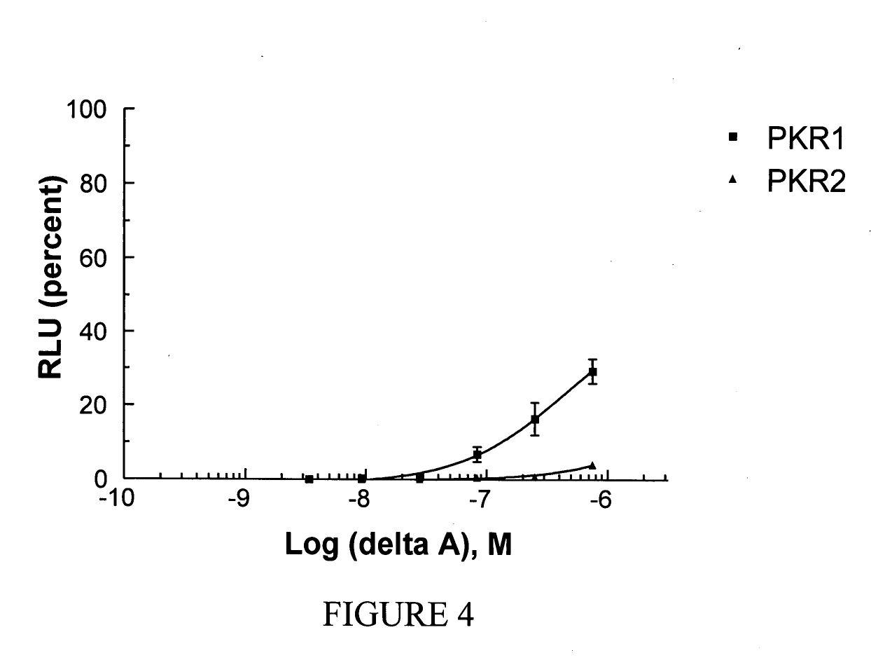

FIG. 4 shows a dose response curve of prokineticin receptor antagonist delA-PK1 (SEQ ID NO: 16) assayed for its ability to activate PKR1- and PKR2-mediated calcium mobilization.

TABLE 2
Structures of Modified Prokineticins

| Name | Structure |
|---|---|
| Wild type | PK1 and PK2 |
| Chimera 12 | AVITG-exon 2 of PK1-exon3 of PK2 |
| Chimera 21 | AVITG-exon2 of PK2-exon3 of PK1 |
| PK2-insert | Insertion of 23 amino acids between exon2 |
| | and exon 3 |
| C18S | Substitute cysteine 18 of PK1 with serine |
| C60R | Substitute cysteine 60 of PK1 with |
| | arginine |
| AVITG- | Fuse AVITG to the N-terminus of colipase |
| colipase | |
| AVITG- | Fuse AVITG to the N-terminus of dickkopf |
| dickkopf | |
| DelA | Delete the alanine 1 of PK1 |
| MV PK1 | Substitute alanine 1 of PK1 with |
| | methionine |
| Met PK1 | Add a methionine to the N-terminus of PK1 |
| GIL-PK1 | Add a tripeptide Gly-Ile-Leu to the N- |
| | terminus of PK1 |
| Ala6 | Mutate the N-terminal AVITGA of PK1 to |
| | AAAAAA |
| Peptide | AVITGACERDVQCG |

These data and other data obtained using similar methods (see also Example III) show that (a) modified prokineticins C18S, C60R, AVITG-colipase, AVITG-dickkopf, MV PK1, Met PK1 and Ala6, lack detectable agonist activity, (b) modified prokineticin GIL-PK1 has weak agonist activity, (c) chimera 12 and 21 have agonist activity, (d) PK2-insert has partial agonist activity and (e) Met PK1 and MV PK1 have antagonist activity.

### EXAMPLE II

### Determination of the Ability of PK Receptor Antagonists to Inhibit Endothelial Cell Proliferation

This example describes a method for determining the ability of a PK receptor antagonist to reduce proliferation of endothelial cells.

Methods for culturing endothelial cells have been described. Luteal endothelial cells (LEC) from microvessels of the bovine corpus luteum are purified as described by Spanel-Borowski and Van-der-Bosch (Cell Tissue Res, 261: 35-47(1990)). Briefly, endothelial cells are dislodged from developing corpora lutea by mechanical dissection followed by collagenase digestion and separated by Percoll density centrifugation. The endothelial cells (1×10⁵ cells/well) are grown in RPM1 1640 containing 10% FCS, 1 mM L-glutamine, 10 mM Na-pyruvate, 100 U/ml penicillin, and 100 ug/ml streptomycin on plates precoated with collagen type I.

Bovine adrenocortical endothelial cells (ACE) are prepared by enzymatic and mechanical dispersion from the adrenal cortex, as described (Homsby P J, et al., “Culturing steroidogenic cells,” Methods in Enzymology, 206:371-380 (1991)). Briefly, bovine adrenal glands are extensively washed with ice cold Ringer solution and perfused through the adrenal vein for 20 minutes with 0.25% collagenase in Ringer solution at 37° C. The glands are then homogenized, and the digested material suspended in Percoll and centrifuged at 13,000 revolutions/minute in an angle-head SS-34 rotor on a Sorvall RCRB centrifuge. The band containing the highest density of ACE is plated in 35 mm petri dishes (Nunc; Roskilde, Denmark) at a cell density of 5×10⁵ cells per dish. ACE relative density in the cell mixture is increased by differential plating. This technique takes advantage of the strong adhesion of ACE to plastic to remove chromaffin and other cells by shaking the culture dish and washing with culture medium 2-4 hours after plating. Freshly dissociated. ACE cells are placed in medium 199 supplemented with 20% fetal calf serum, 2 mM glutamine, 50 U/ml penicillin, and 50 ug/ml streptomycin (Biofluids; Rockville, Md.). Primary cell suspensions are stored frozen in liquid nitrogen. Frozen cells are thawed and plated in Dulbecco's modified Eagle's medium (DMEM)/Ham's F-12 1:1 with 10% fetal bovine serum, 10% horse serum and 0.1 ng/ml recombinant basic fibroblast growth factor (Mallinckrodt; St. Louis, Mo.).

MS1 cell lines are cultured in DMEM as described (Arbiser et al., Proc. Natl. Acad. Sci., 94:861-866, (1997)).

For proliferation assays, 5000 cells/per well endothelial cells (ACE, LEC or MS1) are plated in 24-well dishes. Negative controls include wells in the basic assay media without added factors.

Various concentrations of PK and PK receptor antagonist are tested. Treatments include PK1 (5 nM); and PK1 (5 nM)+PK receptor antagonist (0, 0.3, 1, 3, 16, 30, 100, 300 and 1000 nM). Endothelial cells are counted 5 to 7 days after culturing.

In summary, this example shows that the effect of a PK receptor antagonist on endothelial cell proliferation can be determined using primary or cultured endothelial cells.

### EXAMPLE III

### Prokineticin Receptor Antagonists Reduce Prokineticin Receptor-Mediated Cell Growth

This example shows the ability of prokineticin receptor antagonists to reduce prokineticin receptor 1 mediated cell growth.

To determine whether prokineticin receptor antagonists MetPK1 and MV PK1 have the ability to modulate PK receptor function, the modified prokineticins were tested for their ability to function as antagonists in PK receptor-mediated cell growth inhibition assays. Shown in Table 2, above, are the structures of MetPK1 and MV PK1.

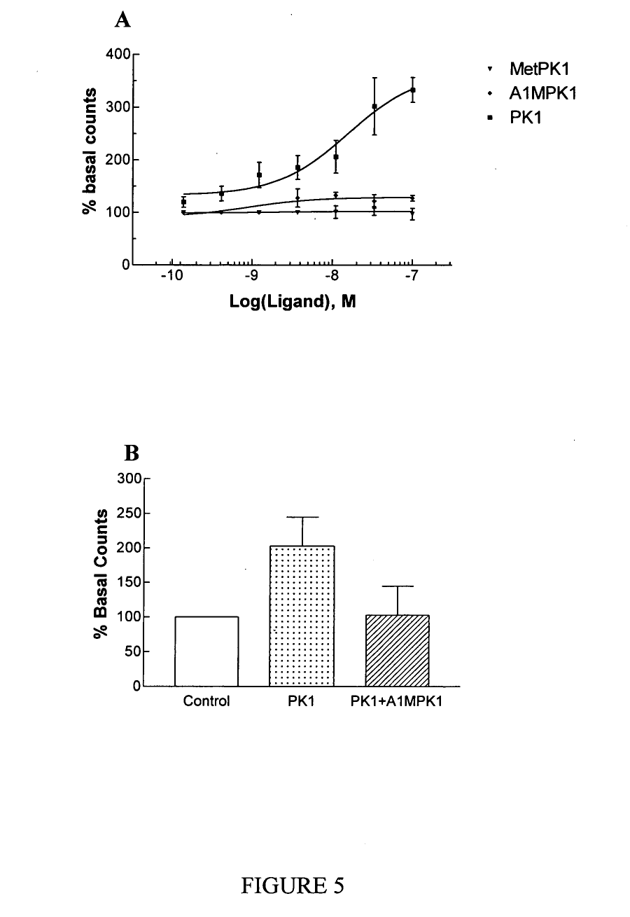

Thymidine incorporation assays in CHO cells stably expressing PKR1 were used to confirm the inhibitory activity of MetPK1 and MV PK1 on PK1-induced PKR1 activity. CHO cells stably expressing human PKR1 were seeded at 5×10⁵ cells per well in 24 well plates. After 36 hours, the cells were placed in serum-free medium for 16 hours. Recombinant PK and PK receptor antagonist polypeptides were then added at various concentrations and allowed to incubate for 8 hours, followed by addition of 5 μCi/ml of [³H] thymidine (76 Ci/mmol) for a further 16 hours. Cells were then washed with 1 ml of ice cold PBS, and 1 ml of ice cold 5% tricholoracetic acid was added. After a 30 minute incubation at 4° C., the cells were washed once with PBS, lysed with 0.5 ml of 0.5 M NaOH/0.5% SDS and counted using a scintillation counter. Results in FIG. 5 are shown as a percentage of basal counts and represent the average ±S.E. of three independent experiments performed in duplicate.

FIG. 5A shows the antagonistic effect of MetPK1 (▾) and MV PK1 (♦)(200 nM) on PK1 (▪)(30 nM)-induced thymidine incorporation. FIG. 5B shows that MV PK1 (striped bar) (200 nM) abolished the PK1 (shaded bar) (30 nM)-induced proliferation activity. Control sample is shown as a colorless bar. These results indicate that PK1 is effective in inducing cell growth and confirm that PK receptor antagonists are effective in inhibiting this biological activity of PK1.

### EXAMPLE IV

### Prokineticin Receptor Antagonists Function as Competitive Antagonists of PKR1 anf PKR2

This example shows that prokineticin receptor antagonists MetPK1 and MV PK1 function as competitive antagonists.

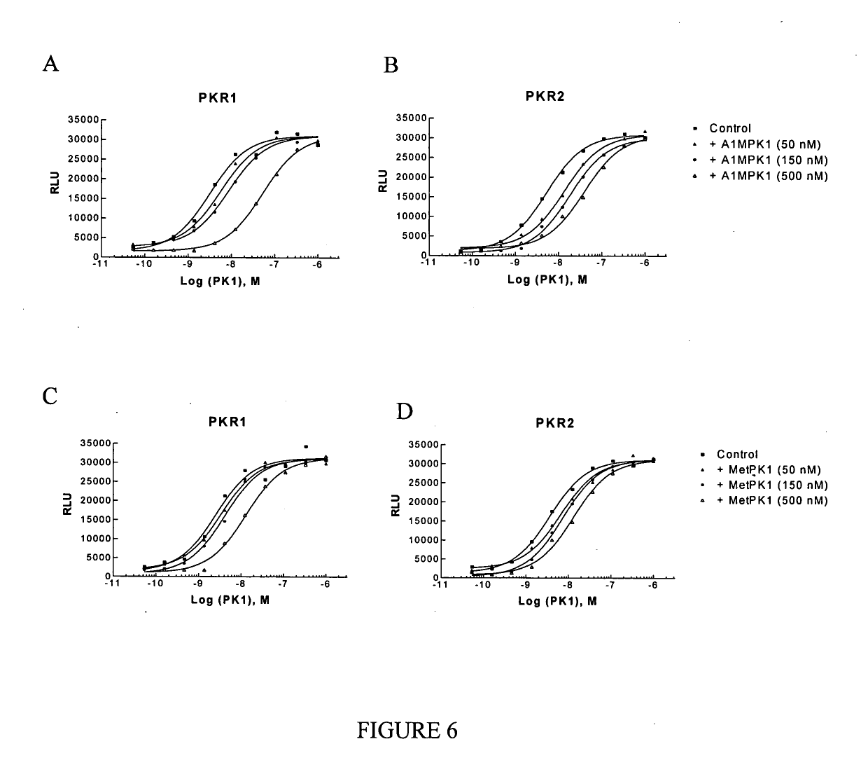

Schild analyses were performed to determine whether MetPK1 and MV PK1 function as competitive antagonists of PKR1 and PKR2. The antagonist activities of MetPK1 and MV PK1 were measured in CHO/AEQ cells that stably express human PKR1 or PKR2. Representative dose-response curves of PK1 in the presence of increasing concentrations of MetPK1 and MV PK1 (50, 150 and 500 nM) are shown in FIG. 6. FIG. 6A shows PKR1 activity in response to PK1 in the presence of 50 (▴), 150 (●) and 500 (Δ) nM MV PK1. FIG. 6B shows PKR2 activity in response to PK1 in the presence of 50 (▴), 150 (●) and 500 (Δ) nM MV PK1. FIG. 6C shows PKR1 activity in response to PK1 in the presence of 50 (▴), 150 (●) and 500 (Δ) nM MetPK1. FIG. 6D shows PKR2 activity in response to PK1 in the presence of 50 (▴), 150 (●) and 500 (Δ) nM MetPK1. These studies revealed that in the presence of increasing concentration of MetPK1 or MV PK1, the dose-response curves of PK1 were shifted to the right, but without change in maximum response. Thus, both MetPK1 and MV PK1 are competitive antagonists for PKR1 and PKR2. The dissociation constants (Kb) of MetPK1 for PKR1 and PKR2 were 260.7±135 nM (n=3) and 48.9±32.1 nM (n=3), respectively. The dissociation constants (Kb) of MV PK1 for PKR1 and PKR2 were 116.1±27.2 nM (n=3) and 37.8±10.5 nM (n=3), respectively.

Throughout this application various publications have been referenced within parentheses. The disclosures of these publications in their entireties are hereby incorporated by reference in this application in order to more fully describe the state of the art to which this invention pertains.

Although the invention has been described with reference to the disclosed embodiments, those skilled in the art will readily appreciate that the specific experiments detailed are only illustrative of the invention. It should be understood that various modifications can be made without departing from the spirit of the invention.

What is claimed is:

1. A method of modulating angiogenesis, comprising administering an amount of a prokineticin receptor antagonist effective to alter one or more indicia of angiogenesis, wherein said antagonist comprises an amino acid sequence at least 80% identical to amino acids to 7 to 77 of SEQ ID NO:3, said sequence comprising;
(a) the 10 conserved cysteine residues of SEQ ID NO:3, and
(b) from 0 to 9 of amino acids 78 to 86 of SEQ ID NO:3,
wherein amino acids 1 to 6 of said antagonist do not consist of amino acids AVITGA (SEQ ID NO:21).

2. The method of claim 1, wherein said antagonist comprises 6 or more amino acids N-terminal to the first conserved cysteine residue.

3. The method of claim 1, wherein said antagonist comprises 7 or more amino acids N-terminal to the first conserved cysteine residue.

4. The method of claim 3, wherein said 7 or more amino acids are MAVITGA (SEQ ID NO:23).

5. The method of claim 4, wherein said antagonist comprises SEQ ID NO:18.

6. The method of claim 5, wherein said antagonist consists of SEQ ID NO:18.

7. The method of claim 2, wherein said 6 or more amino acids are MVITGA (SEQ ID NO:39).

8. The method of claim 7, wherein said antagonist comprises SEQ ID NO:20.

9. The method of claim 8, wherein said antagonist consists of SEQ ID NO:20.

10. The method of claim 1, wherein said antagonist comprises 5 or fewer amino acids N-terminal to said first conserved cysteine residue.

11. The method of claim 10, wherein said 5 or fewer amino acids are VITGA (SEQ ID NO:22).

12. The method of claim 11, wherein said antagonist comprises SEQ ID NO:16.

13. The method of claim 12, wherein said antagonist consists of SEQ ID NO:16.

14. The method of claim 1, wherein amino acid residues that differ from residues 7 to 77 of SEQ ID NO:3 are conservative substitutions thereof.

15. The method of claim 1, wherein amino acid residues that differ from residues 7 to 77 of SEQ ID NO:3 consist of the corresponding residues from SEQ ID NO:6.

16. The method of claim 1, wherein said antagonist comprises amino acids 7 to 77 of SEQ ID NO:3.

17. The method of claim 1, wherein said antagonist is administered to an endothelial cell.

18. The method of claim 1, wherein said one or more indicia of angiogenesis comprises altered cell migration.

19. The method of claim 1, wherein said one or more indicia of angiogenesis comprises altered cell survival.

20. The method of claim 1, wherein said one or more indicia of angiogenesis comprises altered cell morphology.

21. The method of claim 1, wherein said antagonist is administered to a tissue.

22. The method of claim 21, wherein said tissue is any of cornea, chick chorioallantoic membrane and tumor tissue.

23. The method of claim 1, wherein said antagonist is administered to an animal.

24. The method of claim 23, wherein said animal is any of chicken, non-human primate, rat, mouse and human.

25. The method of claim 24, wherein said animal is a human.

26. The method of claim 23, wherein said antagonist is administered to an animal having an angiogenesis-dependent disease.

27. The method of claim 26, wherein said angiogenesis-dependent disease is cancer.

28. A method of modulating angiogenesis, comprising administering an amount of a prokineticin receptor antagonist effective to alter one or more indicia of angiogenesis, wherein said antagonist comprises an amino acid sequence at least 80% identical to amino acids to 7 to 77 of SEQ ID NO:6, said sequence comprising;
(a) the 10 conserved cysteine residues of SEQ ID NO:6, and
(b) from 0 to 4 of amino acids 78 to 81 of SEQ ID NO:6,
wherein amino acids 1 to 6 of said antagonist do not consist of amino acids AVITGA (SEQ ID NO:21).

29. The method of claim 28, wherein said antagonist comprises 6 or more amino acids N-terminal to the first conserved cysteine residue.

30. The method of claim 28, wherein said antagonist comprises 7 or more amino acids N-terminal to the first conserved cysteine residue.

31. The method of claim 30, wherein said 7 or more amino acids are MAVITGA (SEQ ID NO:23).

32. The method of claim 31, wherein said antagonist comprises SEQ ID NO:18.

33. The method of claim 28, wherein said antagonist comprises 5 or fewer amino acids N-terminal to said first conserved cysteine residue.

34. The method of claim 33, wherein said 5 or fewer amino acids are VITGA (SEQ ID NO:22).

35. The method of claim 29, wherein said 6 or more amino acids are MVITGA (SEQ ID NO:39).

36. The method of claim 28, wherein amino acid residues that differ from residues 7 to 77 of SEQ ID NO:6 are conservative substitutions thereof.

37. The method of claim 28, wherein amino acid residues that differ from residues 7 to 77 of SEQ ID NO:6 consist of the corresponding residues from SEQ ID NO:3.

38. The method of claim 28, wherein said antagonist comprises amino acids 7 to 77 of SEQ ID NO:6.

39. The method of claim 28, wherein said antagonist is administered to an endothelial cell.

40. The method of claim 28, wherein said one or more indicia of angiogenesis comprises altered cell migration.

41. The method of claim 28, wherein said one or more indicia of angiogenesis comprises altered cell survival.

42. The method of claim 28, wherein said one or more indicia of angiogenesis comprises altered cell morphology.

43. The method of claim 28, wherein said antagonist is administered to a tissue.

44. The method of claim 43, wherein said tissue is any of cornea, chick chorioallantoic membrane and tumor tissue.

45. The method of claim 28, wherein said antagonist is administered to an animal.

46. The method of claim 45, wherein said animal is any of chicken, non-human primate, rat, mouse and human.

47. The method of claim 45, wherein said antagonist is administered to an animal having an angiogenesis-dependent disease.

48. The method of claim 47, wherein said angiogenesis-dependent disease is cancer.


SEQUENCE LISTING (39 sequences)

SEQ ID NO: 1 - DNA, 1377 bp, Homo sapiens
  CDS (55)...(369)
ggggaagcgagaggcatctaagcaggcagtgttttgccttcaccccaagtgaccatgagaggtgccacgcgagtctcaatcatgctcctcctagtaactgtgtctgactgtgctgtgatcacaggggcctgtgagcgggatgtccagtgtggggcaggcacctgctgtgccatcagcctgtggcttcgagggctgcggatgtgcaccccgctggggcgggaaggcgaggagtgccaccccggcagccacaaggtccccttcttcaggaaacgcaagcaccacacctgtccttgcttgcccaacctgctgtgctccaggttcccggacggcaggtaccgctgctccatggacttgaagaacatcaatttttaggcgcttgcctggtctcaggatacccaccatccttttctgagcacagcctggatttttatttctgccatgaaacccagctcccatgactctcccagtccctacactgactaccctgatctctcttgtctagtacgcacatatgcacacaggcagacatacctcccatcatgacatggtccccaggctggcctgaggatgtcacagcttgaggctgtggtgtgaaaggtggccagcctggttctcttccctgctcaggctgccagagaggtggtaaatggcagaaaggacattccccctcccctccccaggtgacctgctctctttcctgggccctgcccctctccccacatgtatccctcggtctgaattagacattcctgggcacaggctcttgggtgcattgctcagagtcccaggtcctggcctgaccctcaggcccttcacgtgaggtctgtgaggaccaatttgtgggtagttcatcttccctcgattggttaactccttagtttcagaccacagactcaagattggctcttcccagagggcagcagacagtcaccccaaggcaggtgtagggagcccagggaggccaatcagccccctgaagactctggtcccagtcagcctgtggcttgtggcctgtgacctgtgaccttctgccagaattgtcatgcctctgaggccccctcttaccacactttaccagttaaccactgaagcccccaattcccacagcttttccattaaaatgcaaatggtggtggttcaatctaatctgatattgacatattagaaggcaattagggtgtttccttaaacaactcctttccaaggatcagccctgagagcaggttggtgactttgaggagggcagtcctctgtccagattggggtgggagcaagggacagggagcagggcaggggctgaaaggggcactgattcagaccagggaggcaactacacaccaacctgctggctttagaataaaagcaccaactgaactg

SEQ ID NO: 2 - protein, 105 aa, Homo sapiens
MRGATRVSIMLLLVTVSDCAVITGACERDVQCGAGTCCAISLWLRGLRMCTPLGREGEECHPGSHKVPFFRKRKHHTCPCLPNLLCSRFPDGRYRCSMDLKNINF

SEQ ID NO: 3 - protein, 86 aa, Homo sapiens
AVITGACERDVQCGAGTCCAISLWLRGLRMCTPLGREGEECHPGSHKVPFFRKRKHHTCPCLPNLLCSRFPDGRYRCSMDLKNINF

SEQ ID NO: 4 - DNA, 1406 bp, Homo sapiens
  CDS (10)...(333)
gagggcgccatgaggagcctgtgctgcgccccactcctgctcctcttgctgctgccgccgctgctgctcacgccccgcgctggggacgccgccgtgatcaccggggcttgtgacaaggactcccaatgtggtggaggcatgtgctgtgctgtcagtatctgggtcaagagcataaggatttgcacacctatgggcaaactgggagacagctgccatccactgactcgtaaagttccattttttgggcggaggatgcatcacacttgcccatgtctgccaggcttggcctgtttacggacttcatttaaccgatttatttgtttagcccaaaagtaatcgctctggagtagaaaccaaatgtgaatagccacatcttacctgtaaagtcttacttgtgattgtgccaaacaaaaaatgtgccagaaagaaatgctcttgcttcctcaactttccaagtaacatttttatctttgatttgtaaatgatttttttttttttttttatcgaaagagaattttacttttggatagaaatatgaagtgtaaggcattatggaactggttcttatttccctgtttgtgttttggtttgatttggcttttttcttaaatgtcaaaaacgtacccattttcacaaaaatgaggaaaataagaatttgatattttgttagaaaaacttttttttttttttctcaccaccccaagccccatttgtgccctgccgcacaaatacacctacagcttttggtcccttgcctcttccacctcaaagaatttcaaggctcttaccttactttatttttgtccatttctcttccctcctcttgcattttaaagtggagggtttgtctctttgagtttgatggcagaatcactgatgggaatccagctttttgctggcatttaaatagtgaaaagagtgtatatgtgaacttgacactccaaactcctgtcatggcacggaagctaggagtgctgctggacccttcctaaacctgtcactcaagaggacttcagctctgctgttgggctggtgtgtggacagaaggaatggaaagccaaattaatttagtccagatttctaggtttgggtttttctaaaaataaaagattacatttacttcttttactttttataaagttttttttccttagtctcctacttagagatattctagaaaatgtcacttgaagaggaagtatttattttaatctggcacaacactaattaccatttttaaagcggtattaagttgtaatttaaaccttgtttgtaactgaaaggtcgattgtaatggattgccgtttgtacctgtatcagtattgctgtgtaaaaattctgtatcagaataataacagtactgtatatcatttgatttattttaatattatatccttatttttgtc

SEQ ID NO: 5 - protein, 108 aa, Homo sapiens
MRSLCCAPLLLLLLLPPLLLTPRAGDAAVITGACDKDSQCGGGMCCAVSIWVKSIRICTPMGKLGDSCHPLTRKVPFFGRRMHHTCPCLPGLACLRTSFNRFICLAQK

SEQ ID NO: 6 - protein, 81 aa, Homo sapiens
AVITGACDKDSQCGGGMCCAVSIWVKSIRICTPMGKLGDSCHPLTRKVPFFGRRMHHTCPCLPGLACLRTSFNRFICLAQK

SEQ ID NO: 7 - protein, 21 aa, Homo sapiens
NNFGNGRQERRKRKRSKRKKE

SEQ ID NO: 8 - protein, 21 aa, Homo sapiens
SHVANGRQERRRAKRRKRKKE

SEQ ID NO: 9 - protein, 19 aa, Homo sapiens
MRGATRVSIMLLLVTVSDC

SEQ ID NO: 10 - protein, 26 aa, Homo sapiens
MRSLCCAPLLLLLLLPLLLTPPAGDA

SEQ ID NO: 11 - protein, 96 aa, Bombina variegata
MKCFAQIVVLLLVIAFSHGAVITGACDKDVQCGSGTCCAASAWSRNIRFCIPLGNSGEDCHPASHKVPYDGKRLSSLCPCKSGLTCSKSGEKFKCS

SEQ ID NO: 12 - protein, 81 aa, Dendroaspis polylepis polylepis
AVITGACERDLQCGKGTCCAVSLWIKSVRVCTPVGTSGEDCHPASHKIPFSGQRKMHHTCPCAPNLACVQTSPKKFKCLSK

SEQ ID NO: 13 - protein, 81 aa, Artificial Sequence
  synthetic construct
AVITGACERDVQCGAGTCCAISLWLRGLRMCTPLGREGEECHPGSHKVPFFGRRMHHTCPCLPGLACLRTSFNRFICLAQK

SEQ ID NO: 14 - protein, 86 aa, Artificial Sequence
  synthetic construct
AVITGACDKDSQCGGGMCCAVSIWVKSIRICTPMGKLGDSCHPLTRKVPFFRKRKHHTCPCLPNLLCSRFPDGRYRCSMDLKNINF

SEQ ID NO: 15 - protein, 89 aa, Artificial Sequence
  synthetic construct
GILAVITGACERDVQCGAGTCCAISLWLRGLRMCTPLGREGEECHPGSHKVPFFRKRKHHTCPCLPNLLCSRFPDGRYRCSMDLKNINF

SEQ ID NO: 16 - protein, 85 aa, Artificial Sequence
  synthetic construct
VITGACERDVQCGAGTCCAISLWLRGLRMCTPLGREGEECHPGSHKVPFFRKRKHHTCPCLPNLLCSRFPDGRYRCSMDLKNINF

SEQ ID NO: 17 - protein, 86 aa, Artificial Sequence
  synthetic construct
AAAAAACERDVQCGAGTCCAISLWLRGLRMCTPLGREGEECHPGSHKVPFFRKRKHHTCPCLPNLLCSRFPDGRYRCSMDLKNINF

SEQ ID NO: 18 - protein, 87 aa, Artificial Sequence
  synthetic construct
MAVITGACERDVQCGAGTCCAISLWLRGLRMCTPLGREGEECHPGSHKVPFFRKRKHHTCPCLPNLLCSRFPDGRYRCSMDLKNINF

SEQ ID NO: 19 - protein, 14 aa, Artificial Sequence
  synthetic construct
AVITGACERDVQCG

SEQ ID NO: 20 - protein, 86 aa, Homo sapiens
MVITGACERDVQCGAGTCCAISLWLRGLRMCTPLGREGEECHPGSHKVPFFRKRKHHTCPCLPNLLCSRFPDGRYRCSMDLKNINF

SEQ ID NO: 21 - protein, 6 aa, Homo sapiens
AVITGA

SEQ ID NO: 22 - protein, 5 aa, Homo sapiens
VITGA

SEQ ID NO: 23 - protein, 7 aa, Homo sapiens
MAVITGA

SEQ ID NO: 24 - protein, 393 aa, Homo sapiens [sequence omitted]

SEQ ID NO: 25 - protein, 384 aa, Homo sapiens
MAAQNGNTSFTPNFNPPQDHASSLSFNFSYGDYDLPMDEDEDMTKTRTFFAAKIVIGIALAGIMLVCGIGNFVFIAALTRYKKLRNLTNLLIANLAISDFLVAIICCPFEMDYYVVRQLSWEHGHVLCASVNYLRTVSLYVSTNALLAIAIDRYLAIVHPLKPRMNYQTASFLIALVWMVSILIAIPSAYFATETVLFIVKSQEKIFCGQIWPVDQQLYYKSYFLFIFGVEFVGPVVTMTLCYARISRELWFKAVPGFQTEQIRKRLRCRRKTVLVLMCILTAYVLCWAPFYGFTIVRDFFPTVFVKEKHYLTAFYVVECIAMSNSMINTVCFVTVKNNTMKYFKKMMLLHWRPSQRGSKSSADLDLRTNGVPTTEEVDCIRLK

SEQ ID NO: 26 - protein, 393 aa, Mus musculus
METTVGALGENTTDTFTDFFSALDGHEAQTGSLPFTFSYGDYDMPLDEEEDVTNSRTFFAAKIVIGMALVGIMLVCGIGNFIFITALARYKKLRNLTNLLIANLAISDFLVAIVCCPFEMDYYVVRQLSWEHGHVLCASVNYLRTVSLYVSTNALLAIAIDRYLAIVHPLRPRMKCQTAAGLIFLVWSVSILIAIPAAYFTTETVLVIVERQEKIFCGQIWPVDQQFYYRSYFLLVFGLEFVGPVVAMTLCYARVSRELWFKAVPGFQTEQIRRTVRCRRRTVLGLVCVLSAYVLCWAPFYGFTIVRDFFPSVFVKEKHYLTAFYVVECIAMSNSMINTLCFVTVRNNTSKYLKRILRLQWRASPSGSKASADLDLRTTGIPATEEVDCIRLK

SEQ ID NO: 27 - protein, 381 aa, Mus musculus
MGPQNRNTSFAPDLNPPQDHVSLNYSYGDYDLPLGEDEDVTKTQTFFAAKIVIGVALAGIMLVCGIGNFVFIAALARYKKLRNLTNLLIANLAISDFLVAIVCCPFEMDYYVVRQLSWAHGHVLCASVNYLRTVSLYVSTNALLAIAIDRYLAIVHPLKPRMNYQTASFLIALVWMVSILIAVPSAYFTTETILVIVKNQEKIFCGQIWSVDQQLYYKSYFLFVFGLEFVGPVVTMTLCYARISQELWFKAVPGFQTEQIRKRLRCRRKTVLLLMGILTAYVLCWAPFYGFTIVRDFFPTVVVKEKHYLTAFYVVECIAMSNSMINTICFVTVKNNTMKYFKKMLRLHWRPSHYGSKSSADLDLKTSGVPATEEVDCIRLK

SEQ ID NO: 28 - protein, 86 aa, Mus musculus
AVITGACERDIQCGAGTCCAISLWLRGLRLCTPLGREGEECHPGSHKIPFLRKRQHHTCPCSPSLLCSRFPDGRYRCFRDLKNANF

SEQ ID NO: 29 - protein, 81 aa, Mus musculus
AVITGACDKDSQCGGGMCCAVSIWVKSIRICTPMGQVGDSCHPLTRKVPFWGRRMHHTCPCLPGLACLRTSFNRFICLARK

SEQ ID NO: 30 - protein, 86 aa, Rattus sp.
AVITGACERDVQCGAGTCCAISLWLRGLRLCTPLGREGEECHPGSHKIPFFRKRQHHTCPCSPSLLCSRFPDGRYRCSQDLKNVNF

SEQ ID NO: 31 - protein, 81 aa, Rattus sp.; identical to SEQ ID NO: 29

SEQ ID NO: 32 - protein, 77 aa, Bombina maxima
AVITGACDRDVQCGSGTCCAASLWSRNIRFCVPLGNNGEECHPASHKVPYNGKRLSSLCPCKSGLTCSKSGEKFQCS

SEQ ID NO: 33 - DNA, 1204 bp, Macaca mulatta
  CDS (5)...(329)
cgccatgaggagcctgtgctgcgccccactcctgctcctcctgctgctgccgccgctgctgctcacgccccgcgtcggggacgccgccgtgatcaccggggcttgtgacaaggactcccaatgtggtggaggcatgtgctgtgctgtcagtatctgggttaagagcataaggatttgcacacctatgggcaaactgggagacagctgccatccactgactcgtaaagttccatttgttgggcggaggatgcatcacacttgcccatgtctgccaggcttggcctgtttacggacttcatttaaccgatttatttgtttagcccgaaagtaatcgctttaaagtagaaaccaaatgtgaatagccacatcttatctgtaaagtcttacttgtgattgtgccaaacaaaaaatgtgccagaaagaaatgcttttgcttcctcaactttccaagtaacttttttatctttgagttttaaatgattttttttttaatcgggaattttacttttggatagaaatataaagtgtaaggcattgtggaactggttctcatttccctgtttgtgttttggtttggtttggcttttttcttaaatgtcaaaaacatacccattttcacaaaaatgaggaaaataggaatttgatattttgttagagaaactttttttttcctcaccatcccaagccccatttgtgccccgccacaccataccatacatacatacatacatacatacatacatacatacaacttttggtcccttgcctcttccacctcaaagaatttcaaggcccttaccttactttatttttctccatttctcttccctgctcttgcattttaaagtggtaggtttatctctttgagtttgatggcagaatcgctgatgggaatccagctttttgccggctatttaaatagtgaaaagagtttatatgtgaacttgacactccaaactcctctcatggcgtggacgctgggagtgctgccggacccttcctaaacctgtcactcaagaggacttcggctctgctgttgggctggtgtgtggacagaaggaatggaaagctaaattaatttagtccagatttctaggtttgggtttttctaaaaatgaaagattacgtttacttctttttctttttataaagtttttttttcttagtctcctacttagagatattctagaaaatgtcacttgaa

SEQ ID NO: 34 - protein, 108 aa, Macaca mulatta
MRSLCCAPLLLLLLLPPLLLTPRVGDAAVITGACDKDSQCGGGMCCAVSIWVKSIRICTPMGKLGDSCHPLTRKVPFVGRRMHHTCPCLPGLACLRTSFNRFICLARK

SEQ ID NO: 35 - DNA, 1155 bp, Pan troglodyte
  CDS (1)...(1155)
atggcagcccagaatggaaacaccagtttcgcacccaactttaatccaccgcaagaccatgcctcctccctctcctttaacttcagttatggtgattatgacctccctatggatgaggatgaggacatgaccaagacccggaccttcctcgcagccaagatcgtcgttggcattgcactggcaggcatcatgctggtctgcggcatcggtaactttgtctttatcgctgccctcacccgctataagaagttgcgcaacctcaccaatctgctcattgccaacctggccatctccgacttcctggtggccatcatctgctgccccttcgagatggactactacgtggtacggcagctctcctgggagcatggccacgtgctctgtgcctccgtcaactacctgcgcaccgtctccctctacgtctccaccaatgccttgctggccatcgccattgacagatatctcgccattgttcaccctttgaaaccacggatgaattatcaaacggcctccttcctgatcgccttggtctggatggtgtccattctcattgccatcccatcggcctactttgcaacagaaaccgtcctctttattgtcaagagccaggagaagatcttctgtggccagatctggcccgtggatcagcagctctactacaagtcctacttcctcttcatctttggtgtcgagttcgtgggccctgtggtcaccatgaccctgtgctatgccaggatctcccgggagctctggttcaaggcagtccctgggttccagacggagcagattcgcaagcggctgcgctgccgcaggaagacggtcctggtgctcatgtgcattctcacggcctatgtgctgtgctgggcacccttctacggtttcaccatcgttcgtgacttcttccccactgtgttcgtgaaggaaaagcactacctcactgccttctacgtggtcgagtgcatcgccatgagcaacagcatgatcaacaccgtgtgcttcgtgacggtcaagaacaacaccatgaagtacttcaagaagatgatgctgctgcactggcgtccctcccagcgggggagcaagtccagtgccgaccttgacctcagaaccaacggggtgcccgccacagaagaggtggactgtatcaggctgaagtga

SEQ ID NO: 36 - protein, 384 aa, Pan troglodyte
MAAQNGNTSFAPNFNPPQDHASSLSFNFSYGDYDLPMDEDEDMTKTRTFLAAKIVVGIALAGIMLVCGIGNFVFIAALTRYKKLRNLTNLLIANLAISDFLVAIICCPFEMDYYVVRQLSWEHGHVLCASVNYLRTVSLYVSTNALLAIAIDRYLAIVHPLKPRMNYQTASFLIALVWMVSILIAIPSAYFATETVLFIVKSQEKIFCGQIWPVDQQLYYKSYFLFIFGVEFVGPVVTMTLCYARISRELWFKAVPGFQTEQIRKRLRCRRKTVLVLMCILTAYVLCWAPFYGFTIVRDFFPTVFVKEKHYLTAFYVVECIAMSNSMINTVCFVTVKNNTMKYFKKMMLLHWRPSQRGSKSSADLDLRTNGVPATEEVDCIRLK

SEQ ID NO: 37 - DNA, 1155 bp, Saimiri sciureus
  CDS (1)...(1155)
atggcagcccagaatggaaacaccagttttgcacccaactttaatccaccccaagaccatgcctcctccctctccttcaacttcagttatggtgattacgacctccctatggatgaggatgaggacatgaccaagacccggaccttctttgcagccaagattgtcatcggcattgcactggcaggcatcatgctggtctgtggtgtcggtaactttgtctttatcgctgccctcacccgctataagaagctgcgcaacctcaccaatctgctcattgccaacctggccatctccgacttcctggtggccatcatctgctgcccctttgagatggactactatgtggtccggcagctctcctgggagcatggccacgtgctctgtgcctctgtcaactacctgcgcaccgtctccctctacgtctccaccaatgccttgctggccatcgccattgacagatatctcgccattgttcaccccttgaaaccaaggatgaattatcaaacggcctccttcctgatcgccttggtctggatggtatccattctcattgccatcccatcagcctactttgcaacagaaaccgtcctctttattgtcaagagccaggagaagatcttctgtggccagatctggcccgtggatcagcagctctactacaagtcctacttcctcttcatctttggtgtggagttcgtgggtcctgtggtcaccatgaccctgtgctacgccaggatttcccaggagctctggttcaaggcagtccctgggttccagacagagcagatccgtaagcggctgcgctgccgcaggaagacagtcctggtgctcatgtgcatcctcatggcctacgtgctatgctgggcacccttctatggtttcaccatcgtacgcgacttcttccccaccgtgttcgtaaaggaaaagcactacctcactgccttctacgtggtcgagtgcatcgccatgagcaacagcatgatcaacaccgtgtgcttcgtgacggtcaagaacaacaccatgaagtatttcaagaagatgatgctgctgcactggcgtccctcccagcgggggagcaagtccagtgccgaccttgaccttaagacgaacggggtgcctgccacggaagaggtggactgtatcaggctgaagtga

SEQ ID NO: 38 - protein, 384 aa, Saimiri sciureus
MAAQNGNTSFAPNFNPPQDHASSLSFNFSYGDYDLPMDEDEDMTKTRTFFAAKIVIGIALAGIMLVCGVGNFVFIAALTRYKKLRNLTNLLIANLAISDFLVAIICCPFEMDYYVVRQLSWEHGHVLCASVNYLRTVSLYVSTNALLAIAIDRYLAIVHPLKPRMNYQTASFLIALVWMVSILIAIPSAYFATETVLFIVKSQEKIFCGQIWPVDQQLYYKSYFLFIFGVEFVGPVVTMTLCYARISQELWFKAVPGFQTEQIRKRLRCRRKTVLVLMCILMAYVLCWAPFYGFTIVRDFFPTVFVKEKHYLTAFYVVECIAMSNSMINTVCFVTVKNNTMKYFKKMMLLHWRPSQRGSKSSADLDLKTNGVPATEEVDCIRLK

SEQ ID NO: 39 - protein, 6 aa, Artificial Sequence
  synthetic construct
MVITGA